(19) United States
(12) Patent Application Publication
(10) Pub. No.: US 2016/0206546 A1
(43) Pub. Date: Jul. 21, 2016

(54) COMPOSITION COMPRISING AN AMINOSILICONE, A CATIONIC SURFACTANT, AN ASSOCIATIVE POLYMER AND A THICKENING AGENT

(71) Applicant: L'OREAL, Paris (FR)

(72) Inventors: Pedro Aprigliano FERNANDES, Rio de Janeiro (BR); Fabiana PINHO, Rio de Janeiro (BR)

(21) Appl. No.: 14/908,897

(22) PCT Filed: Aug. 1, 2013
(86) PCT No.: PCT/BR2013/000297

§ 371 Date: Jan. 29, 2016

(51) Int. Cl. A61K 8/898; A61K 8/73; A61K 8/43; A45D 20/22; A61K 8/365; A61K 8/41; A61K 8/90; A45D 2/00; A61Q 5/12; A61K 8/34

(52) U.S. Cl. CPC A61K 8/898; A61Q 5/12; A61K 8/731; A61K 8/43; A61K 8/342; A61K 8/365; A61K 8/416; A61K 8/90; A45D 2/001; A45D 20/22; A61K 2800/594; A61K 2800/548; A61K 2800/5424; A61K 2800/5422; A61K 2800/5426; A61K 2800/5428; A61K 2800/48

(57) ABSTRACT

Composition comprising an aminosilicone, a cationic surfactant, an associative polymer and a thickening agent The present invention relates to a composition for treating keratin fibres, in particular human keratin fibres such as the hair, comprising (i) one or more amino silicones, (ii) one or more cationic surfactants different from (i), (iii) one or more associative polymers and (iv) one or more thickening agents different from (iii). The invention also concerns a process for treating keratin fibres, in particular human keratin fibres such as the hair, and a use for hair care employing the said composition.

The present invention relates to a composition for treating keratin fibres, in particular human keratin fibres such as the hair, comprising (i) one or more amino silicones, (ii) one or more cationic surfactants different from (i), (iii) one or more associative polymers and (iv) one or more thickening agents different from (iii).

The invention also concerns a process for treating keratin fibres, in particular human keratin fibres such as the hair, and a use for hair care employing the said composition.

Many people are unsatisfied with the way their hair looks, and have difficulty in styling it. Hair is generally damaged and embrittled by the action of external atmospheric agents such as light and bad weather, and also by mechanical or chemical treatments, such as brushing, combing, dyeing, bleaching, permanent-waving and/or relaxing.

Hair is thus damaged by these various factors and may over time become dry, coarse or dull, especially in fragile areas, and more particularly at the ends leading to the apparition of split ends.

Thus, to overcome these drawbacks, it is common practice to resort to hair care products using compositions that condition the hair appropriately, giving it satisfactory cosmetic properties, especially in terms of smoothness, sheen, softness, suppleness, lightness, a natural feel and good disentangling properties, while at the same time being capable of minimizing the split ends.

These hair care compositions may be, for example, conditioning shampoos, hair conditioners, masks or sera, and may be in the form of gels, hair lotions or care creams that are more or less thick.

These compositions may comprise silicones, which are used as conditioning agents, so as to give the hair a satisfactory level of care, especially in terms of softness, smoothness and suppleness, and to manage the reduction of split ends.

However, silicones often have the drawback of making the hair very lank and heavy, which leads to the phenomenon commonly known as the “build-up effect”. In other words, silicones become deposited in a large amount of material on the hair, which has the consequence of making the head of hair lank and of limiting the use of silicones for conditioning the hair.

These hair care products may also the drawback of giving to the hair an unnatural aspect.

Therefore, there is a real need to develop compositions, for example hair conditioners, that do not have the combination of drawbacks described above, i.e. which are capable of treating and positively nourishing the hair, especially sensitized hair, namely by affording it softness, smoothness and suppleness while at the same time leaving it more light and natural.

The Applicant has discovered, surprisingly, that it is possible to formulate compositions for the treatment of keratin fibres, which have the desired properties, by combining in these compositions one or more amino silicones, one or more cationic surfactants, one or more associative polymers and one or more thickening agents different from the associative polymers previously mentioned.

In particular, it has been found that such a combination allows depositing selectively a suitable amount of amino silicones on hair, especially on sensitized hair, in order to improve the cosmetic properties conferred to it while at the same time leaving it more light and natural. In other words, the treated hair is less lank and heavy while the cosmetic properties are improved.

More particularly, the deposits of silicones lead to softer, smoother and suppler hair and the number of splits ends are also reduced.

The present invention relates especially to a composition comprising:

(i) one or more amino silicones,

(ii) one or more cationic surfactants different from (i),

(iii) one or more associative polymers, and

(iv) one or more thickening agents different from (iii).

The composition of the present invention is able to improve the cosmetic properties of keratin fibres, in particular human keratin fibres such as the hair, especially in terms of softness, smoothness and suppleness while at same time giving the hair a more natural look and feeling.

The present invention also relates to a process for treating the hair, comprising the application to the said fibres of the composition according to the invention.

The invention also relates to the use of the composition according to the invention as a hair conditioner, preferably used after the application of a shampoo.

Other subjects and characteristics, aspects and advantages of the invention will emerge even more clearly on reading the description and the example that follows.

Amino Silicone

The term “amino silicone” according to the present invention means any polyaminosiloxane, i.e. any polysiloxane comprising at least one primary, secondary or tertiary amine function or a quaternary ammonium group.

Preferably, the amino silicone(s) used in the composition according to the present invention are chosen from:

(a) the compounds corresponding to formula (I) below:

(R¹)ₐ(T)₃₋ₐ-Si[OSi(T)₂]ₙ-[OSi(T)_{b}(R¹)_{2-b}]ₘ—OSi(T)₃₋ₐ-(R¹)ₐ (I)

in which:

T is a hydrogen atom or a phenyl, hydroxyl (—OH) or C₁-C₈ alkyl radical, and preferably methyl, or a C₁-C₈ alkoxy, preferably methoxy,

a denotes the number 0 or an integer from 1 to 3, and preferably 0,

b denotes 0 or 1, and in particular 1,

m and n are numbers such that the sum (n+m) can range especially from 1 to 2000 and in particular from 50 to 150, it being possible for n to denote a number from 0 to 1999 and in particular from 49 to 149, and for m to denote a number from 1 to 2000 and in particular from 1 to 10;

R¹ is a monovalent radical of formula —C_{q}H_{2q}L in which q is a number from 2 to 8 and L is an optionally quaternized amino group chosen from the following groups:

—N(R²)—CH₂—CH₂—N(R²)₂;

—N(R²)₂; —N⁺(R²)₃ Q⁻;

—N⁺(R²) (H)₂ Q⁻;

—N⁺(R²)₂HQ⁻;

—N(R²)—CH₂—CH₂—N⁺(R²)(H)₂ Q⁻,

in which R² denotes a hydrogen atom, a phenyl, a benzyl or a saturated monovalent hydrocarbon-based radical, for example a C₁-C₂₀ alkyl radical, and Q⁻ represents a halide ion, for instance fluoride, chloride, bromide or iodide.

In a particular embodiment, the amino silicones corresponding to the definition of formula (I) are chosen from the compounds corresponding to formula (IA) below:

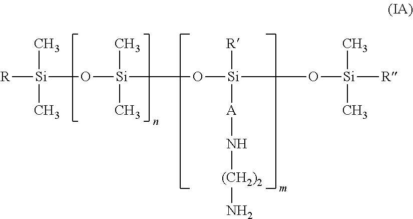

in which R, R′ and R″, which may be identical or different, denote a C₁-C₄ alkyl radical, preferably CH₃; a C₁-C₄ alkoxy radical, preferably methoxy; or OH; A represents a linear or branched, C₃-C₈ and preferably C₃-C₆ alkylene radical; m and n are integers dependent on the molecular weight and whose sum is between 1 and 2000.

According to a first embodiment, R, R′ and R″, which may be identical or different, represent a C₁-C₄ alkyl or hydroxyl radical, A represents a C₃ alkylene radical and m and n are such that the weight-average molecular mass of the compound is between 5000 and 500 000 approximately. Compounds of this type are referred to in the CTFA dictionary as “amodimethicones”.

According to a second embodiment, R, R′ and R″, which may be identical or different, represent a C₁-C₄ alkoxy or hydroxyl radical, at least one of the radicals R or R″ is an alkoxy radical and A represents a C₃ alkylene radical. The hydroxy/alkoxy mole ratio is preferably between 0.2/1 and 0.4/1 and advantageously equal to 0.3/1. Moreover, m and n are such that the weight-average molecular mass of the compound is between 2000 and 10⁶. More particularly, n is between 0 and 999 and m is between 1 and 1000, the sum of n and m being between 1 and 1000.

In this category of compounds, mention may be made, inter alia, of the product Belsil® ADM 652 sold by Wacker.

According to a third embodiment, R and R″, which are different, represent a C₁-C₄ alkoxy or hydroxyl radical, at least one of the radicals R or R″ is an alkoxy radical, R′ represents a methyl radical and A represents a C₃ alkylene radical. The hydroxy/alkoxy mole ratio is preferably between 1/0.8 and 1/1.1 and advantageously equal to 1/0.95. Moreover, m and n are such that the weight-average molecular mass of the compound is between 2000 and 200 000. More particularly, n is between 0 and 999 and m is between 1 and 1000, the sum of n and m being between 1 and 1000.

More particularly, mention may be made of the product Fluid WR® 1300 sold by Wacker.

According to a fourth embodiment, R and R″ represent a hydroxyl radical, R′ represents a methyl radical and A is a C₄-C₈ and preferably C₄ alkylene radical. Moreover, m and n are such that the weight-average molecular mass of the compound is between 2000 and 10⁶. More particularly, n is between 0 and 1999 and m is between 1 and 2000, the sum of n and m being between 1 and 2000.

A product of this type is especially sold under the name DC 28299 by Dow Corning.

Note that the molecular mass of these silicones is determined by gel permeation chromatography (ambient temperature, polystyrene standard; μ styragem columns; eluent THF; flow rate 1 mm/m; 200 μl of a solution containing 0.5% by weight of silicone in THF are injected, and detection is performed by refractometry and UV-metry).

A product corresponding to the definition of formula (IA) is in particular the polymer known in the CTFA dictionary (7th edition, 1997) as “trimethylsilyl amodimethicone”, corresponding to formula (IB) below:

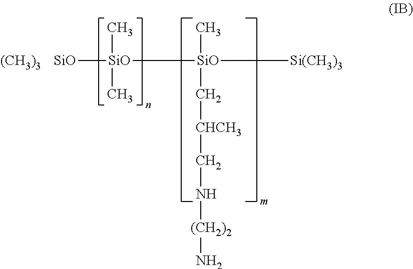

in which n and m have the meanings given above in accordance with formula (I) or (IA).

Such compounds are described, for example, in EP 0 095 238; a compound of formula (IB) is sold, for example, under the name Q2-8220 by the company OSI.

(b) the compounds corresponding to formula (II) below:

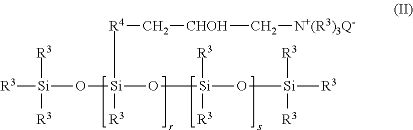

in which:

R³ represents a C₁-C₁₈ monovalent hydrocarbon-based radical, and in particular a C₁-C₁₈ alkyl or C₂-C₁₈ alkenyl radical, for example methyl;

R⁴ represents a divalent hydrocarbon-based radical, especially a C₁-C₁₈ alkylene radical or a divalent C₁-C₁₈, and for example C₁-C₈, alkylenoxy radical;

Q⁻ is a halide ion, in particular chloride;

r represents a mean statistical value from 2 to 20 and in particular from 2 to 8;

s represents a mean statistical value from 20 to 200 and in particular from 20 to 50.

Such compounds are described more particularly in U.S. Pat. No. 4,185,087.

A compound falling within this class is the product sold by the company Union Carbide under the name Ucar Silicone ALE 56.

(c) the quaternary ammonium silicones of formula (III):

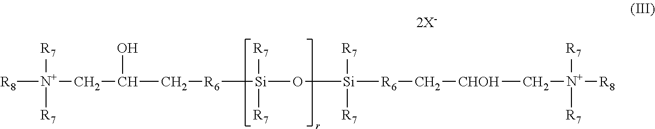

in which:

R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring comprising 5 or 6 carbon atoms, for example methyl;

R₆ represents a divalent hydrocarbon-based radical, especially a C₁-C₁₈ alkylene radical or a divalent C₁-C₁₈, and for example C₁-C₈, alkylenoxy radical linked to the Si via an SiC bond;

R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl radical or a radical —R₆—NHCOR₇;

X⁻ is an anion such as a halide ion, especially chloride, or an organic acid salt (acetate, etc.);

r represents a mean statistical value from 2 to 200 and in particular from 5 to 100;

These silicones are described, for example, in patent application EP-A 0 530 974.

(d) the amino silicones of the following formula (IV):

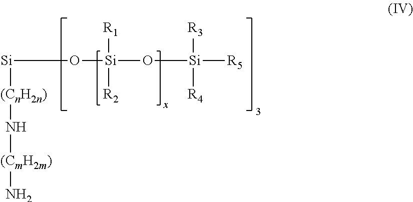

in which:
R₁, R₂, R₃ and R₄, which may be identical or different, denote a C₁-C₄ alkyl radical or a phenyl group,
R₅ denotes a C₁-C₄ alkyl radical or a hydroxyl group,
n is an integer ranging from 1 to 5,
m is an integer ranging from 1 to 5, and

in which x is chosen such that the amine number is between 0.01 and 1 meq/g.

(e) multiblock polyoxyalkylenated amino silicones, of the type (AB)n, A being a polysiloxane block and B being a polyoxyalkylene block comprising at least one amine group.

The said silicones preferably consist of repeating units having the following general formulae:

[—(SiMe₂O)ₓSiMe₂-R—N(R″)—R′—O(C₂H₄O)ₐ(C₃H₆O)_{b}—R′—N(H)—R—]

or alternatively

[—(SiMe₂O)ₓSiMe₂-R—N(R″)—R′—O(C₂H₄O)ₐ(C₃H₆O)_{b}—]

in which:
a is an integer greater than or equal to 1, preferably ranging from 5 to 200 and more particularly ranging from 10 to 100;
b is an integer between 0 and 200, preferably ranging from 4 to 100 and more particularly ranging from 5 to 30;
x is an integer ranging from 1 to 10 000 and more particularly from 10 to 5000;
R″ is a hydrogen atom or a methyl;
R, which may be identical or different, represent a linear or branched C₂-C₁₂ divalent hydrocarbon-based radical, optionally comprising one or more heteroatoms such as oxygen; preferably, R denotes an ethylene radical, a linear or branched propylene radical, a linear or branched butylene radical, or a —CH₂CH₂CH₂OCH(OH)CH₂— radical; preferentially, R denotes a —CH₂CH₂CH₂OCH(OH)CH₂— radical;
R′, which may be identical or different, represent a linear or branched C₂-C₁₂ divalent hydrocarbon-based radical, optionally comprising one or more heteroatoms such as oxygen; preferably, R′ denotes an ethylene radical, a linear or branched propylene radical, a linear or branched butylene radical, or a —CH₂CH₂CH₂OCH(OH)CH₂— radical; preferentially, R denotes —CH(CH₃)—CH₂—.

The siloxane blocks preferably represent from 50 mol % to 95 mol % of the total weight of the silicone, more particularly from 70 mol % to 85 mol %.

The amine content is preferably between 0.02 and 0.5 meq/g of copolymer in a 30% solution in dipropylene glycol, more particularly between 0.05 and 0.2.

The weight-average molecular weight (Mw) of the silicone is preferably between 5000 and 1 000 000 and more particularly between 10 000 and 200 000.

Mention may be made especially of the silicones sold under the names Silsoft A-843 or Silsoft A+ by Momentive.

The silicone(s) that are particularly preferred are the multiblock polyoxyalkylenated aminated silicones.

The amino silicone(s) used in the composition according to the invention may be present in a content ranging from 0.1% to 15% by weight, preferably in a content ranging from 0.5% to 12% by weight and better still in a content ranging from 1% to 8% by weight relative to the total weight of the composition.

Cationic Surfactant

Cationic surfactants used in the composition of the present invention may be selected from salts of optionally polyoxyalkylenated primary, secondary or tertiary fatty amines, quaternary ammonium salts, and mixtures thereof.

The cationic surfactants used in the composition of the present invention are preferably quaternary ammonium salts.

Quaternary ammonium salts include especially, for example:
those corresponding to the general formula (VI) below:

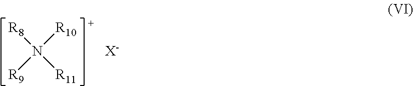

in which the radicals R₈ to R₁₁, which may be identical or different, represent a linear or branched aliphatic radical comprising from 1 to 30 carbon atoms, or an aromatic radical such as aryl or alkylaryl, with at least one of the radicals R₈ to R₁₁ denoting a linear or branched alkyl radical comprising from 10 to 30 carbon atoms, and X— denoting an organic or inorganic anion. The aliphatic radicals may include heteroatoms such as, in particular, oxygen, nitrogen, sulfur and halogens. The aliphatic radicals are selected for example from C₁₋₃₀ alkyl, C₁₋₃₀ alkoxy, polyoxyalkylene (C₂-C₆), C₁₋₃₀ alkylamide, alkyl(C₁₂-C₂₂)amidoalkyl(C₂-C₆) and C₁₋₃₀ hydroxyalkyl radicals; X is an anion selected from the group of halides, phosphates, acetates, lactates, alkyl(C₂-C₆)sulfates and alkyl- or alkylaryl-sulfonates.

Among the quaternary ammonium salts of formula (VI), preference is firstly given to tetraalkylammonium chlorides such as, for example, dialkyldimethylammonium or alkyltrimethylammonium chlorides in which the alkyl radical comprises approximately from 12 to 22 carbon atoms, more particularly behenyltrimethylammonium, distearyldimethylammonium, cetyltrimethylammonium, and benzyldimethylstearylammonium chlorides, or else, secondly, to palmitylamidopropyltrimethylammonium chloride or stearamidopropyldimethyl(myristyl acetate)-ammonium chloride, which is sold under the name Ceraphyl® 70 by the company Van Dyk.
quaternary ammonium salts of imidazoline, such as, for example, those of formula (VII) below:

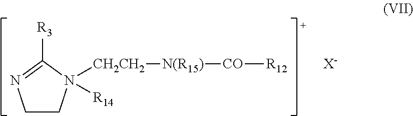

in which R₁₂ represents an alkenyl or alkyl radical comprising from 8 to 30 carbon atoms, derived for example from tallow fatty acids, R₁₃ represents a hydrogen atom, a C₁-C₄ alkyl radical or an alkenyl or alkyl radical comprising from 8 to 30 carbon atoms, R₁₄ represents a C₁-C₄ alkyl radical, R₁₅ represents a hydrogen atom or a C₁-C₄ alkyl radical, X⁻ is an anion selected from the group of halides, phosphates, acetates, lactates, alkyl sulfates and alkyl- or alkylaryl-sulfonates. Preferably, R₁₂ and R₁₃ denote a mixture of alkenyl or alkyl radicals comprising from 12 to 21 carbon atoms, for example derived from tallow fatty acids, R₁₄ denotes a methyl radical and R₁₅ denotes a hydrogen atom. A product of this kind is sold for example under the name Rewoquat® W 75 by the company Rewo;
quaternary diammonium or triammonium salts, particularly of formula (VIII) below:

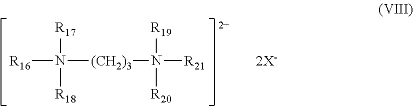

in which formula (VIII):

R₁₆ denotes an alkyl group comprising approximately from 16 to 30 carbon atoms, which is optionally hydroxylated and/or interrupted with one or more oxygen atoms;

R₁₇ is selected from hydrogen, an alkyl group comprising from 1 to 4 carbon atoms or a group —(CH₂)₃—N⁺(R₁₆ₐ)(R₁₇ₐ)(R₁₈ₐ), X⁻;

R₁₆ₐ, R₁₇ₐ, R₁₈ₐ, R₁₈, R₁₉, R₂₀ and R₂₁, which may be identical or different, are selected from hydrogen and an alkyl group comprising from 1 to 4 carbon atoms; and

X⁻, which may be identical or different, represent an organic or inorganic anionic counterion, such as that selected from halides, acetates, phosphates, nitrates, alkyl(C₁-C₄) sulfates, alkyl(C₁-C₄)- or alkyl(C₁-C₄)aryl-sulfonates, more particularly methyl sulfate and ethyl sulfate.

Compounds of this kind are, for example, Finquat CT-P, available from the company Finetex (Quaternium 89), and Finquat CT, available from the company Finetex (Quaternium 75);
quaternary ammonium salts containing one or more ester functions, such as those of formula (IX) below:

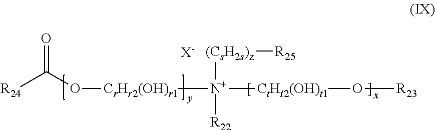

in which formula (IX):

R₂₂ is selected from C₁-C₆ alkyl groups and C₁-C₈ hydroxyalkyl or C₁-C₆ dihydroxyalkyl groups,

R₂₃ is selected from:
the group

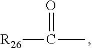

linear or branched, saturated or unsaturated C₁-C₂₂ hydrocarbon groups R₂₇,
hydrogen atom,

R₂₅ is selected from:
the group

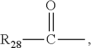

linear or branched, saturated or unsaturated C₁-C₆ hydrocarbon groups R₂₉,
hydrogen atom,

R₂₄, R₂₆ and R₂₈, which are identical or different, are selected from linear or branched, saturated or unsaturated C₇-C₂₁ hydrocarbon groups;

r, s and t, which are identical or different, are integers from 2 to 6,

r1 and t1, which are identical or different, are equal to 0 or 1, with r2+r1=2r and t1+t2=2t,

y is an integer ranging from 1 to 10,

x and z, which are identical or different, are integers from 0 to 10,

X⁻ represents an organic or inorganic anionic counterion,

with the proviso that the sum x+y+z equals from 1 to 15, that, when x is 0, then R₂₃ denotes R₂₇ and that, when z is 0, then R₂₅ denotes R₂₉.

The alkyl groups R₂₂ may be linear or branched, and more particularly linear.

Preferably, R₂₂ denotes a methyl, ethyl, hydroxyethyl or dihydroxypropyl group, and more particularly a methyl or ethyl group.

Advantageously, the sum x+y+z has a value from 1 to 10.

When R₂₃ is an R₂₇ hydrocarbon group, it may be long and may have from 12 to 22 carbon atoms, or may be short and may have from 1 to 3 carbon atoms.

When R₂₅ is an R₂₉ hydrocarbon group, it preferably has 1 to 3 carbon atoms.

Advantageously, R₂₄, R₂₆ and R₂₈, which are identical or different, are selected from linear or branched, saturated or unsaturated C₁₁-C₂₁ hydrocarbon groups, and more particularly from linear or branched, saturated or unsaturated C₁₁-C₂₁ alkyl and alkenyl groups.

Preferably, x and z, which are identical or different, are equal to 0 or 1.

Advantageously, y is 1.

Preferably, r, s and t, which are identical or different, are 2 or 3, and more particularly they are 2.

The anionic counterion X⁻ is preferably a halide, such as chloride, bromide or iodide; a alkyl(C₁-C₄) sulfate or a alkyl(C₁-C₄)- or alkyl(C₁-C₄)aryl-sulfonate. It is possible, however, to use methanesulfonate, phosphate, nitrate or tosylate, an anion derived from organic acid such as acetate or lactate, or any other anion compatible with the ester-functional ammonium.

The anionic counterion X⁻ is even more particularly chloride, methyl sulfate or ethyl sulfate.

Use is made more particularly, in the composition according to the invention, of the ammonium salts of formula (VI) in which:
R₂₂ denotes a methyl or ethyl group,
x and y are equal to 1,
z is equal to 0 or 1,
r, s and t are equal to 2,
R₂₃ is selected from:

the group

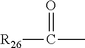

methyl, ethyl or C₁₄-C₂₂ hydrocarbon groups,

a hydrogen atom,
R₂₅ is selected from:

the group

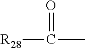

a hydrogen atom,
R₂₄, R₂₆ and R₂₈, which may be identical or different, are selected from linear or branched, saturated or unsaturated C₁₃-C₁₇ hydrocarbon groups, and preferably from linear or branched, saturated or unsaturated C₁₃-C₁₇ alkyl and alkenyl groups.

Advantageously, the hydrocarbon radicals are linear.

Mention may be made, for example, among the compounds of formula (IX), of diacyloxyethyldimethylammonium, diacyloxyethylhydroxyethylmethylammonium, monoacyloxyethyldihydroxyethylmethylammonium, triacyloxyethylmethylammonium and monoacyloxyethylhydroxyethyldimethylammonium salts, especially the chloride or the methyl sulfate, and mixtures of these compounds. The acyl groups preferably have 14 to 18 carbon atoms and originate more particularly from a vegetable oil such as palm oil or sunflower oil. When the compound contains a plurality of acyl groups, these groups may be identical or different.

These products are obtained, for example, by direct esterification of triethanolamine, triisopropanolamine, an alkyldiethanolamine or an alkyldiisopropanolamine, which are optionally oxyalkylenated, with fatty acids or with fatty acid mixtures of plant or animal origin, or by transesterification of the methyl esters thereof. This esterification is followed by quaternization by means of an alkylating agent such as an alkyl halide, preferably methyl or ethyl halide, a dialkyl sulfate, preferably dimethyl or diethyl sulfate, methyl methanesulfonate, methyl para-toluenesulfonate, glycol chlorohydrin or glycerol chlorohydrin.

Such compounds are sold, for example, under the names Dehyquart® by the company Henkel, Stepanquat® by the company Stepan, Noxamium® by the company Ceca, Rewoquat® WE 18 by the company Rewo-Witco.

The composition according to the invention may contain, for example, a mixture of quaternary ammonium monoester, diester and triester salts, with a majority by weight of diester salts.

It is also possible to use the ammonium salts containing at least one ester function that are described in U.S. Pat. No. 4,874,554 and U.S. Pat. No. 4,137,180.

Use may be made of the behenoylhydroxypropyltrimethylammonium chloride available from Kao under the name Quatarmin BTC 131.

Preferably, the ammonium salts containing at least one ester function contain two ester functions.

Among the quaternary ammonium salts containing at least one ester function that can be used, it is preferred to use dipalmitoylethylhydroxyethylmethylammonium salts.

The cationic surfactants used in the composition are preferably selected from quaternary ammonium salts of formula (III) and quaternary ammonium salts comprising at least one ester function, especially those selected from those corresponding to the formula (VI).

The cationic surfactants used in the composition are preferably selected from behenyltrimethylammonium, distearyldimethylammonium, cetyltrimethylammonium and benzyldimethylstearylammonium chlorides, or else from palmitylamido-propyltrimethylammonium or stearamidopropyldimethyl(myristyl acetate)-ammonium chlorides.

More preferably still, the cationic surfactant is cetyltrimethylammonium chloride or behenyltrimethylammonium chloride

Cationic surfactants of the invention are different from ingredients (i). Preferably, they are non siliconated.

The term non siliconated means that there is no siloxane group Si—O in the chemical structure of the surfactant.

The cationic surfactants used in the composition of the present invention may be present in said composition in an amount of from 0.1 wt % to 20% by wt %, more preferably from 0.5 to 15% by wt %, preferably from 1 by wt % to 10 by wt %, relative to the total weight of the composition.

Associative Polymer

Associative polymers are water-soluble or water dispersible polymers that are capable, in an aqueous medium, of reversibly combining with each other or with other molecules.

In one embodiment, the associative polymer used in the invention is an amphiphilic polymer, which means a polymer comprising at least one hydrophilic moiety which renders the polymer soluble in water and at least one hydrophobic region, comprising at least one fatty chain, by means of which the polymer interacts and undergoes assembly with another associative polymer or with other molecules.

Hence the associative polymer used in the present invention is preferably an amphiphilic polymer which comprises at least one hydrophilic group and at least one fatty chain.

According to the present invention, a fatty chain has at least 8 carbon atoms, preferably from 8 to 30 carbon atoms, more preferably from 10 to 30 carbon atoms and especially from 12 to 22 carbon atoms.

According to the present invention the associative polymer has at least one repetitive unit other than an oxyalkylenated group. So associative polymers are different from products resulting merely from the condensation of an alkylene oxide with an alcohol, an ester or an amide.

The associative polymers of the present invention may be chosen from non-ionic, anionic, cationic and amphoteric associative polymers.

Anionic Associative Polymer

The associative polymer may be chosen from anionic amphiphilic polymer.

Among the anionic amphiphilic associative polymers comprising at least one fatty chain, mention may be made of

(I) anionic associative polymers comprising at least one hydrophilic unit and at least one fatty-chain allyl ether unit, such as those whose hydrophilic unit comprises an ethylenic unsaturated anionic monomer, such as vinylcarboxylic acid, acrylic acid, methacrylic acid, and mixtures thereof, wherein the fatty-chain allyl ether unit of which corresponds to the monomer of formula (X) below:

CH₂—CR′CH₂OBnR (X)

in which

R′ is chosen from a hydrogen atom and CH₃;

B is an ethyleneoxy radical;

n is an integer ranging from 0 to 100;

R is a hydrocarbon-based radical chosen from alkyl, arylalkyl, aryl, alkylaryl, and cycloalkyl radicals, having from 8 to 30 carbon atoms, such as from 10 to 24 carbon atoms, or from 12 to 18 carbon atoms. A unit of formula (I) that may be mentioned is a unit in which R′ is a hydrogen atom, n is equal to 10, and R is a stearyl (C18) radical.

Anionic amphiphilic polymers of this type are described and prepared, for example, according to an emulsion polymerization process in patent EP 0 216 479.

Among these fatty-chain anionic associative polymers, those that may be mentioned according certain embodiments are polymers formed from at least one of 20% to 60% by weight of acrylic acid and methacrylic acid, from 5% to 60% by weight of at least one lower alkyl (meth)acrylate, from 2% to 50% by weight of at least one fatty-chain allyl ether of formula (X), and from 0% to 1% by weight of at least one crosslinking agent which may be a well-known copolymerizable unsaturated polyethylenic monomer, for instance diallyl phthalate, allyl (meth)acrylate, divinylbenzene, (poly)ethylene glycol dimethacrylate, and methylenebisacrylamide.

Among the latter polymers, those according to certain embodiments include crosslinked terpolymers of methacrylic acid, of ethyl acrylate and of polyethylene glycol (10 EO) stearyl ether alcohol (Steareth-10), such as those sold by the company Allied Colloids under the names SALCARE® SC 80 and SALCARE® SC 90, which are aqueous 30% emulsions of a crosslinked terpolymer of methacrylic acid, of ethyl acrylate, and of steareth-10 allyl ether, each unit comprising 40%, 50%, and 10%, respectively.

(II) anionic associative polymers comprising at least one unsaturated olefinic carboxylic acid hydrophilic unit, and at least one unsaturated carboxylic acid (C₁₀-C₃₀)alkyl ester hydrophobic unit. These polymers may be chosen from those in which the unsaturated olefinic carboxylic acid hydrophilic unit corresponds to the monomer of formula (XI) below:

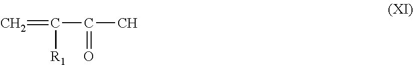

in which R₁₆ is chosen from a hydrogen atom, CH₃, and C₂H₅, that is to say acrylic acid, methacrylic acid, and ethacrylic acid units, and in which the unsaturated carboxylic acid (C₁₀-C₃₀)alkyl ester hydrophobic unit corresponds to the monomer of formula (XII) below:

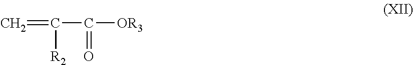

in which

R₁₇ is chosen from a hydrogen atom, CH₃, and C₂H₅, that is to say acrylate, methacrylate, and ethacrylate units, for example hydrogen, i.e., acrylate units, and CH₃, i.e., methacrylate units; and R₁₈ is a C₁₀-C₃₀, such as a C₁₂-C₂₂ alkyl radical.

(C₁₀-C₃₀) alkyl esters of unsaturated carboxylic acids according to certain embodiments may include, for example, lauryl acrylate, stearyl acrylate, decyl acrylate, isodecyl acrylate, dodecyl acrylate, and the corresponding methacrylates, lauryl methacrylate, stearyl methacrylate, decyl methacrylate, isodecyl methacrylate, and dodecyl methacrylate.

Anionic polymers of this type are described and prepared, for example, according to U.S. Pat. Nos. 3,915,921 and 4,509,949.

Among the anionic associative polymers of this type, those that may be mentioned include polymers formed from a monomer mixture comprising:

(i) at least one acrylic acid;

(ii) at least one ester of formula (XII) described above in which R₁₇ is chosen from a hydrogen atom and CH₃, and R₁₈ is chosen from alkyl radicals having from 12 to 22 carbon atoms;

(iii) and at least one crosslinking agent, which may be a well-known copolymerizable polyethylenic unsaturated monomer, for instance diallyl phthalate, allyl (meth)acrylate, divinylbenzene, (poly)ethylene glycol dimethacrylate, and methylenebisacrylamide.

Among fatty-chain anionic associative polymers of this type, those that may be mentioned include those comprising from 60% to 95% by weight of acrylic acid as the hydrophilic unit, from 4% to 40% by weight of C₁₀-C₃₀ alkyl acrylate as the hydrophobic unit, and from 0% to 6% by weight of crosslinking polymerizable monomer, or alternatively those comprising from 96% to 98% by weight of acrylic acid as the hydrophilic unit, from 1% to 4% by weight of C₁₀-C₃₀ alkyl acrylate as the hydrophobic unit, and from 0.1% to 0.6% by weight of crosslinking polymerizable monomer such as those described above.

Among the said above polymers, those that may be mentioned, by way of non-limiting example, are the products sold by the company Goodrich under the trade names PEMULEN® TR1, PEMULEN® TR2, and CARBOPOL® 1382, for example PEMULEN® TR1, and the product sold by the company SEPPIC under the name COATEX Sx.

(III) maleic anhydride/C₃₀-C₃₈ α-olefin/alkyl maleate terpolymers, such as the product comprising maleic anhydride/C₃₀-C₃₈ α-olefin/isopropyl maleate copolymer sold under the name Performa V 1608 by the company Newphase Technologies.

(IV) acrylic terpolymers comprising:

(a) 20% to 70% by weight of a carboxylic acid containing α, β-monoethylenic unsaturation,

(b) 20% to 80% by weight of a non-surfactant monomer containing α, β-monoethylenic unsaturation and being other than (a),

(c) 0.5% to 60% by weight of a non-ionic monourethane which is the product of reaction of a monohydric surfactant with a monoisocyanate containing monoethylenic unsaturation, such as those described in patent application EP A 0 173 109, for example the terpolymer described in Example 3, namely a methacrylic acid/methyl acrylate/ethoxylated (40 EO) behenyl dimethyl-meta-isopropenylbenzylisocyanate terpolymer, as an aqueous 25% dispersion.

(V) copolymers comprising among their monomers a carboxylic acid containing α, β-monoethylenic unsaturation and an ester of a carboxylic acid containing α, β-monoethylenic unsaturation and of an oxyalkylenated fatty alcohol.

These compounds may also comprise as monomer an ester of a carboxylic acid containing α, β-monoethylenic unsaturation and of a C₁-C₄ alcohol.

An example of a compound of this type which may be mentioned is ACULYN® 22 sold by the company Rohm & Haas, which is a methacrylic acid/ethyl acrylate/stearyl methacrylate oxyalkylenated terpolymer.

Non Ionic Associative Polymer

The associative polymer may be chosen from non ionic amphiphilic polymer.

The associative nonionic polymers according to the invention may be chosen from:
(1) celluloses modified with groups comprising at least one fatty chain;

of which, examples that may be mentioned include:
hydroxyethylcelluloses modified with groups comprising at least one fatty chain, such as alkyl, arylalkyl or alkylaryl groups, or mixtures thereof, and in which the alkyl groups are preferably C₈-C₂₂, for instance the product Natrosol Plus Grade 330 CS® (C₁₆ alkyl) sold by the company Aqualon, or the product Bermocoll EHM 100® sold by the company Berol Nobel,
hydroxyethylcelluloses modified with alkylphenyl polyalkylene glycol ether groups, such as the product Amercell Polymer HM-1500® (polyethylene glycol (15) nonylphenyl ether) sold by the company Amerchol,
(2) hydroxypropyl guars modified with groups comprising at least one fatty chain, such as the product Esaflor HM 22® (C₂₂ alkyl chain) sold by the company Lamberti, and the products RE210-18® (C₁₄ alkyl chain) and RE205-1® (C₂₀ alkyl chain) sold by the company Rhône-Poulenc.
(3) copolymers of C₁-C₆ alkyl methacrylates or acrylates and of amphiphilic monomers comprising at least one fatty chain, such as, for example, the oxyethylenated methyl acrylate/stearyl acrylate copolymer sold by the company Goldschmidt under the name Antil 208®.
(4) copolymers of hydrophilic methacrylates or acrylates and of hydrophobic monomers comprising at least one fatty chain, such as, for example, the polyethylene glycol methacrylate/lauryl methacrylate copolymer.
(5) polyurethane polyethers comprising in their chain both hydrophilic blocks usually of polyoxyethylenated nature and hydrophobic blocks, which may be aliphatic sequences alone and/or cycloaliphatic and/or aromatic sequences.
(6) polymers with an aminoplast ether backbone containing at least one fatty chain, such as the Pure Thix® compounds sold by the company Sud-Chemie.

Preferably, the polyurethane polyethers comprise at least two hydrocarbon-based lipophilic chains containing from 8 to 30 carbon atoms, separated by a hydrophilic block, the hydrocarbon-based chains possibly being pendent chains or chains at the end of the hydrophilic block.

In particular, it is possible for one or more pendent chains to be included.

In addition, the polymer may comprise a hydrocarbon-based chain at one end or at both ends of a hydrophilic block.

The polyurethane polyethers may be multiblock, in particular in triblock form. The hydrophobic blocks may be at each end of the chain (for example: triblock copolymer containing a hydrophilic central block) or distributed both at the ends and in the chain (for example multiblock copolymer). These same polymers may also be graft polymers or star polymers.

The fatty-chain nonionic polyurethane polyethers may be triblock copolymers in which the hydrophilic block is a polyoxyethylenated chain comprising from 50 to 1000 oxyethylene groups. The nonionic polyurethane polyethers comprise a urethane linkage between the hydrophilic blocks, whence arises the name.

By extension, also included among the fatty-chain nonionic polyurethane polyethers are those in which the hydrophilic blocks are linked to the lipophilic blocks via other chemical bonds.

As examples of fatty-chain nonionic polyurethane polyethers that may be used in the invention, it is also possible to use Rheolate 205® containing a urea function, sold by the company Rheox, or Rheolate® 208, 204 or 212, and also Acrysol RM 184®.

Mention may also be made of the product Elfacos T210® containing a C₁₂-C₁₄ alkyl chain, and the product Elfacos T212® containing a C₁₈ alkyl chain, from Akzo.

The product DW 1206B® from Rohm & Haas containing a C₂₀ alkyl chain and a urethane bond, sold at a solids content of 20% in water, may also be used.

It is also possible to use solutions or dispersions of these polymers, especially in water or in aqueous-alcoholic medium. Examples of such polymers that may be mentioned are Rheolate® 255, Rheolate® 278 and Rheolate® 244 sold by the company Rheox. The products DW 1206F and DW 1206J sold by the company Röhm & Haas may also be used.

The polyurethane polyethers that may be used according to the invention are in particular those described in the article by G. Fonnum, J. Bakke and Fk. Hansen—Colloid Polym. Sci. 271, 380-389 (1993).

Even more particularly, according to the invention, it is preferred to use a polyurethane polyether that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyurethane polyethers are sold especially by the company Röhm & Haas under the names Aculyn 46® and Aculyn 44® [Aculyn 46® is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%); Aculyn 44® is a polycondensate of polyethylene glycol containing 150 or 180 mol of ethylene oxide, of decyl alcohol and of methylenebis(4-cyclohexyl isocyanate) (SMDI), at 35% by weight in a mixture of propylene glycol (39%) and water (26%)].

Cationic Associative Polymer

The associative polymer may be chosen from cationic amphiphilic polymer.

The associative cationic amphiphilic polymers may be chosen from:
(1) cationic associative amphiphilic polyurethanes, which may be water-soluble or water-dispersible, the family of which has been described by the Applicant in French patent application No. 00/09609.

The cationic associative amphiphilic polyurethanes may be represented by the general formula (XIII) below:

R—X—(P)n-[L-(Y)m]r-L′-(P)p-X′—R′ (XIII)

in which:

R and R′, which may be identical or different, represent a hydrophobic group or a hydrogen atom;

X and X′, which may be identical or different, represent a group comprising an amine function optionally bearing a hydrophobic group, or alternatively a group L″;

L, L′ and L″, which may be identical or different, represent a group derived from a diisocyanate;

P and P′, which may be identical or different, represent a group comprising an amine function optionally bearing a hydrophobic group;

Y represents a hydrophilic group;

r is an integer between 1 and 100, preferably between 1 and 50 and in particular between 1 and 25;

n, m and p each range, independently of each other, between 0 and 1000;

the molecule containing at least one protonated or quaternized amine function and at least one hydrophobic group.

In one preferred embodiment of these polyurethanes, the only hydrophobic groups are the groups R and R′ at the chain ends.

One preferred family of cationic associative polyurethanes is the one corresponding to formula (XIII) described above and in which:

R and R′ both independently represent a hydrophobic group,

X and X′ each represent a group L″,
n and p are between 1 and 1000, and

L, L′, L″, P, P′, Y and m have the meaning given above.

Another preferred family of cationic associative polyurethanes is the one corresponding to formula (I) above in which:

R and R′ both independently represent a hydrophobic group, X and X′ each represent a group L″, n and p are 0, and L, L′, L″, Y and m have the meaning given above.

The fact that n and p are 0 means that these polymers do not comprise units derived from a monomer containing an amine function, incorporated into the polymer during the polycondensation. The protonated amine functions of these polyurethanes result from the hydrolysis of excess isocyanate functions, at the chain end, followed by alkylation of the primary amine functions formed with alkylating agents containing a hydrophobic group, i.e. compounds of the type RQ or R′Q, in which R and R′ are as defined above and Q denotes a leaving group such as a halide, a sulfate, etc.

Yet another preferred family of cationic associative polyurethanes is the one corresponding to formula (I) above in which:

R and R′ both independently represent a hydrophobic group,

X and X′ both independently represent a group comprising a quaternary amine,

n and p are zero, and

L, L′, Y and m have the meaning given above.

The number-average molecular mass of the cationic associative polyurethanes is preferably between 400 and 500 000, in particular between 1000 and 400 000 and ideally between 1000 and 300 000.

The expression “hydrophobic group” means a radical or polymer containing a saturated or unsaturated, linear or branched hydrocarbon-based chain, which may contain one or more heteroatoms such as P, O, N or S, or a radical containing a perfluoro or silicone chain. When the hydrophobic group denotes a hydrocarbon-based radical, it comprises at least 10 carbon atoms, preferably from 10 to 30 carbon atoms, in particular from 12 to 30 carbon atoms and more preferably from 18 to 30 carbon atoms.

Preferentially, the hydrocarbon-based group is derived from a monofunctional compound.

By way of example, the hydrophobic group may be derived from a fatty alcohol such as stearyl alcohol, dodecyl alcohol or decyl alcohol. It may also denote a hydrocarbon-based polymer, for instance polybutadiene.

When X and/or X′ denote(s) a group comprising a tertiary or quaternary amine, X and/or X′ may represent one of the following formulae:

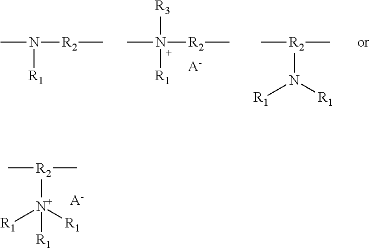

### for X

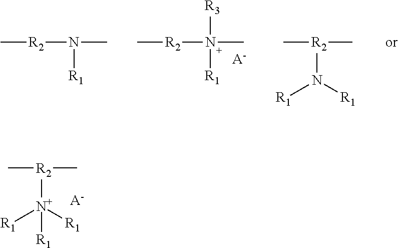

### for X′

in which:

R₂ represents a linear or branched alkylene radical containing from 1 to 20 carbon atoms, optionally comprising a saturated or unsaturated ring, or an arylene radical, one or more of the carbon atoms possibly being replaced with a heteroatom chosen from N, S, O and P;

R₁ and R₃, which may be identical or different, denote a linear or branched C₁-C₃₀ alkyl or alkenyl radical or an aryl radical, at least one of the carbon atoms possibly being replaced with a heteroatom chosen from N, S, O and P;

A⁻ is a physiologically acceptable counterion.

The groups L, L′ and L″ represent a group of formula:

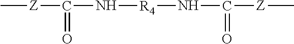

in which:

Z represents —O—, —S— or —NH—; and

R₄ represents a linear or branched alkylene radical containing from 1 to 20 carbon atoms, optionally comprising a saturated or unsaturated ring, or an arylene radical, one or more of the carbon atoms possibly being replaced with a heteroatom chosen from N, S, O and P.

The groups P and P′ comprising an amine function may represent at least one of the following formulae:

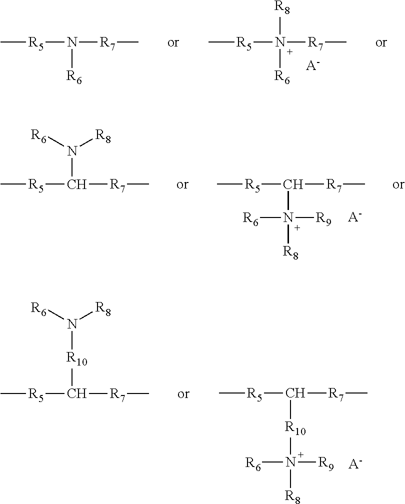

in which:

R₅ and R₇ have the same meanings as R₂ defined above;

R₆, R₈ and R₉ have the same meanings as R₁ and R₃ defined above;

R₁₀ represents a linear or branched, optionally unsaturated alkylene group possibly containing one or more heteroatoms chosen from N, O, S and P; and

A⁻ is a physiologically acceptable counterion.

As regards the meaning of Y, the term “hydrophilic group” means a polymeric or non-polymeric water-soluble group.

By way of example, when it is not a polymer, mention may be made of ethylene glycol, diethylene glycol and propylene glycol.

When it is a hydrophilic polymer, in accordance with one preferred embodiment, mention may be made, for example, of polyethers, sulfonated polyesters, sulfonated polyamides or a mixture of these polymers. The hydrophilic compound is preferentially a polyether and especially a poly(ethylene oxide) or polypropylene oxide).

The cationic associative polyurethanes of formula (XIII) that may be used according to the invention are formed from diisocyanates and from various compounds bearing functions containing a labile hydrogen. The functions containing a labile hydrogen may be alcohol, primary or secondary amine or thiol functions, giving, after reaction with the diisocyanate functions, polyurethanes, polyureas and polythioureas, respectively. The expression “polyurethanes that may be used according to the present invention” encompasses these three types of polymer, namely polyurethanes per se, polyureas and polythioureas, and also copolymers thereof.

A first type of compound involved in the preparation of the polyurethane of formula (XIII) is a compound comprising at least one unit containing an amine function. This compound may be multifunctional, but the compound is preferentially difunctional, that is to say that, according to one preferential embodiment, this compound comprises two labile hydrogen atoms borne, for example, by a hydroxyl, primary amine, secondary amine or thiol function. A mixture of multifunctional and difunctional compounds in which the percentage of multifunctional compounds is low may also be used.

As mentioned above, this compound may comprise more than one unit containing an amine function. In this case, it is a polymer bearing a repetition of the unit containing an amine function.

Compounds of this type may be represented by one of the following formulae:

HZ—(P)n-ZH

or

HZ—(P′)p-ZH

in which Z, P, P′, n and p are as defined above.

Examples of compounds containing an amine function that may be mentioned include N-methyldiethanolamine, N-tert-butyldiethanolamine and N-sulfoethyldiethanolamine.

The second compound included in the preparation of the polyurethane of formula (XIII) is a diisocyanate corresponding to the formula:

O═C═N—R₄—N═C═O

in which R₄ is as defined above.

By way of example, mention may be made of methylenediphenyl diisocyanate, methylenecyclohexane diisocyanate, isophorone diisocyanate, tolylene diisocyanate, naphthalene diisocyanate, butane diisocyanate and hexane diisocyanate.

A third compound involved in the preparation of the polyurethane of formula (XIII) is a hydrophobic compound intended to form the terminal hydrophobic groups of the polymer of formula (XIII).

This compound is formed form a hydrophobic group and a function containing a labile hydrogen, for example a hydroxyl, primary or secondary amine, or thiol function.

By way of example, this compound may be a fatty alcohol such as, in particular, stearyl alcohol, dodecyl alcohol or decyl alcohol. When this compound comprises a polymeric chain, it may be, for example, α-hydroxylated hydrogenated polybutadiene.

The hydrophobic group of the polyurethane of formula (I) may also result from the quaternization reaction of the tertiary amine of the compound comprising at least one tertiary amine unit. Thus, the hydrophobic group is introduced via the quaternizing agent. This quaternizing agent is a compound of the type RQ or R′Q, in which R and R′ are as defined above and Q denotes a leaving group such as a halide, a sulfate, etc.

The cationic associative polyurethane may also comprise a hydrophilic block. This block is provided by a fourth type of compound involved in the preparation of the polymer. This compound may be multifunctional. It is preferably difunctional. It is also possible to have a mixture in which the percentage of multifunctional compound is low.

The functions containing a labile hydrogen are alcohol, primary or secondary amine or thiol functions. This compound may be a polymer terminated at the chain ends with one of these functions containing a labile hydrogen.

By way of example, when it is not a polymer, mention may be made of ethylene glycol, diethylene glycol and propylene glycol.

When it is a hydrophilic polymer, mention may be made, for example, of polyethers, sulfonated polyesters and sulfonated polyamides, or a mixture of these polymers. The hydrophilic compound is preferentially a polyether and especially a polyethylene oxide) or poly(propylene oxide).

The hydrophilic group termed Y in formula (I) is optional. Specifically, the units containing a quaternary amine or protonated function may suffice to provide the solubility or water-dispersibility required for this type of polymer in an aqueous solution.

Although the presence of a hydrophilic group Y is optional, cationic associative polyurethanes comprising such a group are, however, preferred.
(2) quaternized cellulose derivatives,

The quaternized cellulose derivatives are, in particular quaternized celluloses, particularly quaternized hydroxyethylcelluloses modified with groups comprising at least one fatty chain, such as alkyl, arylalkyl or alkylaryl groups comprising at least 8 carbon atoms, or mixtures thereof.

The alkyl radicals borne by the above quaternized celluloses or hydroxyethylcelluloses preferably contain from 8 to 30 carbon atoms, especially from 10 to 30 carbon atoms. The aryl radicals preferably denote phenyl, benzyl, naphthyl or anthryl groups.

Examples of quaternized alkylhydroxyethylcelluloses containing C₈-C₃₀ fatty chains that may be mentioned include the products PQ-67® (C₁₂ alkyl), Quatrisoft LM 200®, Quatrisoft LM-X 529-18-A®, Quatrisoft LM-X 529-18B® (C₁₂ alkyl) and Quatrisoft LM-X-529-8® (C₁₈ alkyl) or Softcat Polymer SL100 (C₁₂ alkyl) sold by the company Amerchol, and the products Crodacel QM®, Crodacel QL® (C₁₂ alkyl) and Crodacel QS® (C₁₈ alkyl) sold by the company Croda.
(3) cationic poly(vinyllactam) polymer which may comprise:
a) at least one monomer of vinyllactam or alkylvinyllactam type;
b) at least one monomer of structure (Ia) or (Ib) below:

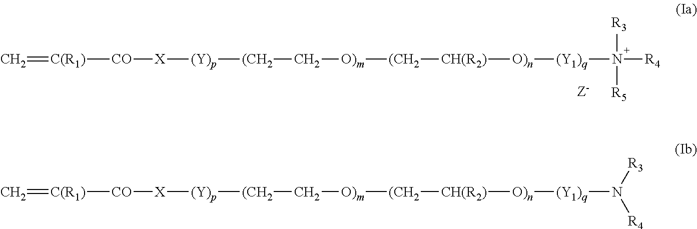

in which:

X denotes an oxygen atom or a radical NR₆,

R₁ and R₅ denote, independently of each other, a hydrogen atom or a linear or branched C₁-C₅ alkyl radical,

R₂ denotes a linear or branched C₁-C₄ alkyl radical,

R₃, R₄ and R₅ denote, independently of each other, a hydrogen atom, a linear or branched C₁-C₃₀ alkyl radical or a radical of formula (II):

—(Y₂)ᵣ—(CH₂—CH(R₇)—O)ₓ—R₈ (II)

Y, Y₁ and Y₂ denote, independently of each other, a linear or branched C₂-C₁₆ alkylene radical,

R₇ denotes a hydrogen atom or a linear or branched C₁-C₄ alkyl radical or a linear or branched C₁-C₄ hydroxyalkyl radical,

R₈ denotes a hydrogen atom or a linear or branched C₁-C₃₀ alkyl radical,

p, q and r denote, independently of each other, either the value 0 or the value 1,

m and n denote, independently of each other, an integer ranging from 0 to 100,

x denotes an integer ranging from 1 to 100,

Z denotes an organic or mineral acid anion,

with the proviso that:
at least one of the substituents R₃, R₄, R₅ or R₈ denotes a linear or branched C₉-C₃₀ alkyl radical,
if m or n is other than zero, then q is equal to 1,
if m or n is equal to zero, then p or q is equal to 0;
at least one fatty alcohol, and
at least one polyol with a molecular weight of greater than 80.

The cationic poly(vinyllactam) polymers used in the composition according to the invention comprise:
a) at least one monomer of vinyllactam or alkylvinyllactam type;
b) at least one monomer of structure (Ia) or (Ib) below:

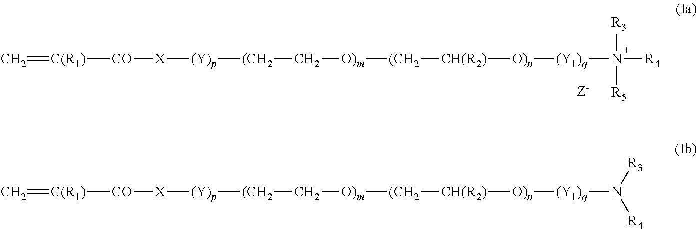

in which:

X denotes an oxygen atom or a radical NR₆,

R₁ and R₆ denote, independently of each other, a hydrogen atom or a linear or branched C₁-C₆ alkyl radical,

R₂ denotes a linear or branched C₁-C₄ alkyl radical,

R₃, R₄ and R₅ denote, independently of each other, a hydrogen atom, a linear or branched C₁-C₃₀ alkyl radical or a radical of formula (XIV):

—(Y₂)ᵣ—(CH₂—CH(R₇)—O)ₓ—R₈ (XIV)

Y, Y₁ and Y₂ denote, independently of each other, a linear or branched C₂-C₁₆ alkylene radical,

R₇ denotes a hydrogen atom or a linear or branched C₁-C₄ alkyl radical or a linear or branched C₁-C₄ hydroxyalkyl radical,

R₈ denotes a hydrogen atom or a linear or branched C₁-C₃₀ alkyl radical,

p, q and r denote, independently of each other, either the value 0 or the value 1,

m and n denote, independently of each other, an integer ranging from 0 to 100,

x denotes an integer ranging from 1 to 100,

Z denotes an organic or mineral acid anion,

with the proviso that:
at least one of the substituents R₃, R₄, R₅ or R₈ denotes a linear or branched C₉-C₃₀ alkyl radical,
if m or n is other than zero, then q is equal to 1,
if m or n is equal to zero, then p or q is equal to 0.

The cationic poly(vinyllactam) polymers used in the composition according to the invention may be crosslinked or noncrosslinked and may also be block polymers.

Preferably, the counterion Z⁻ of the monomers of formula (Ia) is chosen from halide ions, phosphate ions, the methosulfate ion and the tosylate ion.

Preferably, R₃, R₄ and R₅ denote, independently of each other, a hydrogen atom or a linear or branched C₁-C₃₀ alkyl radical.

More preferably, the monomer b) is a monomer of formula (Ia) for which, even more preferably, m and n are equal to 0.

The vinyllactam or alkylvinyllactam monomer is preferably a compound of structure (XV):

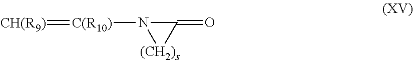

in which:

s denotes an integer ranging from 3 to 6,

R₉ denotes a hydrogen atom or a C₁-C₅ alkyl radical,

R₁₀ denotes a hydrogen atom or a C₁-C₅ alkyl radical,

with the proviso that at least one of the radicals R₉ and R₁₀ denotes a hydrogen atom.

Even more preferably, the monomer (XV) is vinylpyrrolidone.

The cationic poly(vinyllactam) polymers used in the composition according to the invention may also contain one or more additional monomers, preferably cationic or nonionic monomers.

As compounds that are more particularly preferred according to the invention, mention may be made of the following terpolymers comprising at least:

a) one monomer of formula (XV),

b) one monomer of formula (Ia) in which p=1, q=0, R₃ and R₄ denote, independently of each other, a hydrogen atom or a C₁-C₅ alkyl radical and R₅ denotes a C₉-C₂₄ alkyl radical, and

c) one monomer of formula (Ib) in which R₃ and R₄ denote, independently of each other, a hydrogen atom or a C₁-C₅ alkyl radical.

Even more preferentially, terpolymers comprising, by weight, 40% to 95% of monomer (a), 0.1% to 55% of monomer (c) and 0.25% to 50% of monomer (b) will be used.

Such polymers are described in patent application WO 00/68282, the content of which forms an integral part of the invention.

As cationic poly(vinyllactam) polymers according to the invention, vinylpyrrolidone/dimethylaminopropylmethacrylamide/dodecyldimethylmethacryl-amidopropylammonium tosylate terpolymers, vinylpyrrolidone/dimethylaminopropyl-methacrylamide/cocoyldimethylmethacrylamidopropylammonium tosylate terpolymers, vinylpyrrolidone/dimethylaminopropylmethacrylamide/lauryldimethylmethacrylamidopropyl-ammonium tosylate or chloride terpolymers are used in particular.

The weight-average molecular mass of the cationic poly(vinyllactam) polymers according to the present invention is preferably between 500 and 20 000 000. It is more particularly between 200 000 and 2 000 000 and even more preferably between 400 000 and 800 000.

One polymer that is particularly preferred is the polymer sold under the name Styleze W20 by the company ISP, which is a terpolymer of vinylpyrrolidone/dimethylaminopropyl-methacrylamide and of lauryldimethylmethacrylamidopropylammonium chloride.
(4) the cationic polymer(s) obtained by polymerization of a monomer mixture comprising one or more vinyl monomers substituted with one or more amino groups, one or more hydrophobic nonionic vinyl monomers, and one or more associative vinyl monomers.

In particular, among these cationic polymers, mention may be made especially of the compound sold by the company Noveon under the name Aqua CC and which corresponds to the INCI name Polyacrylate-1 Crosspolymer.

Polyacrylate-1 Crosspolymer is the product of polymerization of a monomer mixture comprising:
a di(C₁-C₄ alkyl)amino(C₁-C₆ alkyl) methacrylate,
one or more C₁-C₃₀ alkyl esters of (meth)acrylic acid,
a polyethoxylated C₁₀-C₃₀ alkyl methacrylate (20-25 mol of ethylene oxide units),
a 30/5 polyethylene glycol/polypropylene glycol allyl ether,
a hydroxy(C₂-C₆ alkyl) methacrylate, and
an ethylene glycol dimethacrylate.

In particular, the cationic associative polymer is a cationic amphiphilic polymer having at least one fatty chain comprising at least 8 carbon atoms, especially from 10 to 30 carbon atoms and more specifically from 10 to 22 carbon atoms.

More preferably, the cationic associative polymer is chosen from:
quaternized cellulose particularly quaternized hydroxyethylcelluloses modified with groups comprising at least one fatty chain, such as alkyl, arylalkyl or a alkylaryl groups comprising at least 8 carbon atoms or mixtures thereof,
quaternized hydroxyethylcelluloses modified with groups comprising at least one fatty chain, such as alkyl, arylalkyl or alkylaryl groups comprising at least 8 carbon atoms, or mixtures thereof.

In a more preferred embodiment, the cationic associative polymer is chosen from quaternized hydroxyethylcelluloses modified with at least an alkyl group containing at least 8 carbon atoms, especially from 10 to 22 carbon atoms and more specifically from 10 to 16 carbon atoms.

The cationic associative polymer is especially an associative cationic polymer having the INCI name POLYQUATERNIUM-67.

Amphoteric Associative Polymer

The associative polymer may be chosen from amphoteric amphiphilic polymer.

The amphoteric substantive polymers that may be used in accordance with certain embodiments of the present disclosure may be chosen from polymers comprising units K and M randomly distributed in the polymer chain, in which K denotes a unit derived from a monomer comprising at least one basic nitrogen atom, and M denotes a unit derived from an acidic monomer comprising at least one of carboxylic and sulphonic groups, or alternatively K and M may denote groups derived from monomers chosen from zwitterionic carboxybetaine and sulphobetaine monomers;

K and M may also denote a cationic polymer chain comprising at least one of primary, secondary, tertiary, and quaternary amine groups, in which at least one of the amine groups bears a group chosen from carboxylic and sulphonic groups linked via a hydrocarbon-based radical, or alternatively K and M may form part of a chain of at least one polymer comprising an α,β-dicarboxylic ethylene unit in which one of the carboxylic groups has been made to react with a polyamine comprising at least one of primary and secondary amine groups.

The amphoteric polymers used according to certain embodiments may further comprise at least one fatty chain having 8 to 30 carbon atoms, and may be chosen, for example, from polymers derived from polyaspartic acid and comprising at least one fatty chain having 8 to 30 carbon atoms, such as those:
described and prepared in patent application EP 0 767 191, the content of which is incorporated by reference herein. Such polymers may be prepared in a conventional manner by reacting polysuccinimide (PSI) with fatty-chain (C₈-C₂₄) amines in a solvent medium in the presence or absence of a basic catalyst such as, for example, aliphatic tertiary amines, followed by amphoterization of the resultant product by reaction with a halogenated organic acid.

Among the C₈-C₂₄ fatty-chain amines which may be reacted with the PSI, mention may be made of octylamine, nonylamine, decylamine, dodecylamine, tetradecylamine, hexadecylamine, octadecylamine, octadecenylamine, eicosyldecylamine, octynylamine, decenylamine, dodecenylamine, tetradecenylamine, hexadecenylamine, octadecenylamine, and eicosenylamine.

Examples of such polymers are prepared by reacting PSI with n-laurylamine or with n-stearylamine in the presence of N,N-dimethyl-1,3-propanediamine as basic catalyst, followed by amphoterization of the resultant product by reaction with potassium monochloroacetate. These polymers are prepared with greater details on pages 13 to 20 (lines 1-4) and in Examples 1 to 5 on pages 28 to 34 (lines 14) of the said patent application EP 0 767 191.
described and prepared in patent application EP 0 884 344, the contents of which are incorporated by reference herein. Polymers of this kind may be prepared by reacting gaseous ammonia with a C8-C24 alkyl or alkenyl monomaleate in a solvent medium under reduced pressure at a temperature of 120-140[deg.] C. for from 4 to 6 hours.

The C₈-C₂₄ alkyl or alkenyl radicals may be chosen from linear and branched radicals such as decyl, dodecyl, tetradecyl, hexadecyl, octadecyl, and oleyl radicals.

Examples of such polymers include polymers comprising aspartic acid units and decyl aspartate units, polymers comprising aspartic acid units and dodecyl aspartate units, polymers comprising aspartic acid units and cetyl aspartate units, polymers comprising aspartic acid units and stearyl aspartate units, and polymers comprising aspartic acid units and n-decylaspartamide units, which are described, for example, in Examples 1 to 6 in the said patent application EP 0 884 344;
described and prepared in patent application EP 0 959 094, the contents of which are incorporated by reference herein. Polymers of this kind may be prepared by reacting, in a solvent medium, gaseous ammonia with maleic acid monoamide, polyoxyalkylenated and hydrophobically modified by a at least one of linear or branched C₈-C₃₀ alkyls and alkenyl chains, optionally in a mixture with at least one monoester of maleic acid.

An example of a polymer thus prepared is described in Example 2 on page 11 of the said patent application EP 0 959 094.
described and prepared in patent application EP 0 959 090, the contents of which are incorporated by reference herein. Hydrophobically modified polymers of this kind of high molecular weight may be obtained from derivatives of maleic acid and gaseous ammonia and from difunctional or polyfunctional amines and alcohols.

Examples of copolymers comprising aspartic acid units and cetyl aspartate units and comprising aspartic acid units and cetyl aspartate units are given, respectively, in Examples 3 and 5 of the said patent application EP 0 959 090.
described and prepared in patent application EP 0 959 091, the contents of which are incorporated by reference herein. Hydrophobically modified polymers of this kind may be prepared from at least one of maleic acid monoester, maleic acid monoamide, and gaseous ammonia.

Examples of such copolymers are given in Examples 1, 2, 3, and 5 of the said patent application EP 0 959 091.

In accordance with the present disclosure, the amphoteric polymers comprising at least one fatty chain having 8 to 30 carbon atoms may be chosen from those comprising at least one non-cyclic cationic unit. For example, the amphoteric polymers may be prepared from or comprise from 1 to 20 mol % of monomer comprising a fatty chain, such as from 1.5 to 15 mol % or from 1.5 to 6 mol %, relative to the total number of moles of monomers.

The said fatty-chain amphoteric polymers of which mention may be made comprise, or are prepared by copolymerizing:

1) at least one monomer of formula (XVIa) or (XVIb):

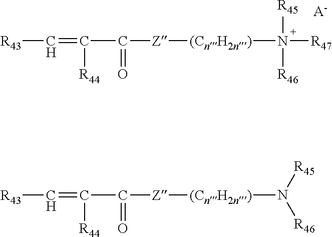

in which R₄₃ and R₄₄, which may be identical or different, are chosen from a hydrogen atom and methyl; R₄₅, R₄₆, and R₄₇, which may be identical or different, are chosen from linear and branched alkyl radicals having from 1 to 30 carbon atoms;

Z″ is chosen from an NH group and an oxygen atom; n′″ is an integer from 2 to 5; and

A⁻ is an anion derived from an acid chosen from organic and mineral acids, such as methosulphate anions and halides such as chloride and bromide;

2) at least one monomer of formula (XVII):

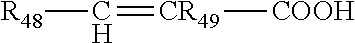

in which R₄₈ and R₄₉, which may be identical or different, are chosen from a hydrogen atom and methyl; and

2) at least one monomer of formula (XVIII):

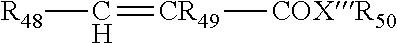

in which R₄₈ and R₄₉, which may be identical or different, are chosen from a hydrogen atom and methyl, X′″ is chosen from oxygen and nitrogen, and R₅₀ is chosen from linear and branched alkyl radicals having from 1 to 30 carbon atoms; wherein at least one of the monomers of formula (XVIa), (XVVb) and (XVII) comprises at least one fatty chain.

The monomers of formulae (XVIa) and (XVIb) disclosed herein may be chosen from:
dimethylaminoethyl methacrylate, dimethylaminoethyl acrylate,
diethylaminoethyl methacrylate, diethylaminoethyl acrylate,
dimethylaminopropyl methacrylate, dimethylaminopropyl acrylate,
dimethylaminopropylmethacrylamide, and dimethylaminopropylacrylamide,

these monomers optionally being quaternized, for example with a C₁-C₄ alkyl halide or a C₁-C₄ dialkyl sulphate.

In according with the present disclosure, the monomer of formula (XVIa) may be chosen from acrylamidopropyltrimethylammonium chloride and methacrylamidopropyltrimethylammonium chloride.

The monomers of formula (XVII) disclosed herein may be chosen from acrylic acid, methacrylic acid, crotonic acid, and 2-methylcrotonic acid. For example, the monomer of formula (XVII) may be acrylic acid.

The monomers of formula (XVIII) disclosed herein may be chosen from C₁₂-C₂₂, such as C₁₆-C₁₈, alkyl acrylates and methacrylates.

The monomers comprising the fatty-chain amphoteric polymers disclosed herein may be already neutralized and/or quaternized.

The ratio of the number of cationic charges to anionic charges is, in certain embodiments, to about 1.

The fatty-chain amphoteric polymers disclosed herein may comprise from 1 mol % to 10 mol % of the monomer comprising a fatty chain, such as monomers of formula (XVIa), (XVIb), and (XVII), for example from 1.5 mol % to 6 mol %.

The weight-average molecular weights of the fatty-chain amphoteric polymers disclosed herein may range from 500 to 50,000,000, such as from 10,000 to 5,000,000.

The fatty-chain amphoteric polymers may also comprise other monomers such as non-ionic monomers, C₁-C₄ alkyl acrylates and methacrylates.

Fatty-chain amphoteric polymers disclosed here are described and prepared, for example, in patent application WO 98/44012.

Among the fatty-chain amphoteric polymers, the ones that may be mentioned are acrylic acid/(meth)acrylamidopropyltrimethylammonium chloride/stearyl methacrylate terpolymers.

In a preferred embodiment, the associative polymer is a cationic amphiphilic polymer.

In particular, the associative polymer is a cationic amphiphilic polymer having at least one fatty chain comprising at least 8 carbon atoms, especially from 10 to 30 carbon atoms and more specifically from 10 to 22 carbon atoms

More preferably, the associative polymer is chosen from:
quaternized celluloses, particularly quaternized hydroxyethylcelluloses, modified with groups comprising at least one fatty chain, such as alkyl, arylalkyl or alkylaryl groups comprising at least 8 carbon atoms, or mixtures thereof,

In a more preferred embodiment, the associative polymer is chosen from quaternized hydroxyethylcelluloses modified with at least an alkyl group containing at least 8 carbon atoms, especially from 10 to 22 carbon atoms and more specifically from 10 to 16 carbon atoms.

The associative polymer is especially an associative cationic polymer having the INCI name POLYQUATERNIUM-67.

The associative(s) polymer(s) used in the present invention may be present in said composition in an amount of from 0.01 wt % to 5 wt %, more preferably from 0.05 to 2 wt %, preferably from 0.1 wt % to 1 wt %, relative to the total weight of the composition.

Thickening Agent

The composition according to the invention further comprises one or more thickeners agents different from the associative polymers previously described.

For the purposes of the present invention, the term “thickener” means any compound whose presence increases the viscosity of the composition into which it is introduced by at least 25 cps and preferably 50 cps at 25° C. and at a shear rate of 1 s⁻¹.

Preferably, the thickener agent according to the invention is non ionic.

The thickener(s) may preferably be chosen from fatty alcohols, fatty acid amides, oxyalkylenated fatty acid esters and thickening polymers, or mixtures thereof.

Fatty Alcohol

For the purposes of the present invention, the term “fatty alcohol” means any saturated or unsaturated, linear or branched alcohol containing at least 8 carbon atoms.

The fatty alcohol may have the structure R—OH in which R denotes a saturated or unsaturated, linear or branched radical containing from 8 to 40 and preferably from 8 to 30 carbon atoms; R preferably denotes a C₈-C₄₀ and preferably C₁₂-C₂₄ alkyl or a C₈-C₄₀ and preferably C₁₂-C₂₄ alkenyl group. R may be substituted with one or more hydroxyl groups and especially with one or two hydroxyl groups.

Examples that may be mentioned include lauryl alcohol, cetyl alcohol, stearyl alcohol, oleyl alcohol, behenyl alcohol, linoleyl alcohol, undecylenyl alcohol, palmitoleyl alcohol, arachidonyl alcohol and erucyl alcohol, and mixtures thereof.

The fatty alcohol may represent a mixture of fatty alcohols, which means that several species of fatty alcohol may coexist, in the form of a mixture, in a commercial product.

Advantageously, the fatty alcohol is solid or pasty at a temperature of 25° C. For the purposes of the present invention, the term “fatty alcohol that is solid or pasty at 25° C.” means a fatty alcohol that has a viscosity, measured with a rheometer (for example an R600 rheometer) at a shear rate of 1 s⁻¹, of greater than or equal to 1 Pa·s.

Preferably, the fatty alcohols used in the composition according to the invention are stearyl alcohol, cetyl alcohol and mixtures thereof, such as cetylstearyl alcohol.

Even more preferentially, the fatty alcohol used in the composition according to the invention is cetyl alcohol.

Fatty Acid Amides

For the purposes of the present invention, the term “fatty acid amide” means an amide comprising in its structure at least one hydrocarbon-based chain comprising at least 8 carbon atoms.

The fatty acid amides are more particularly chosen from compounds derived from an amide of an alkanolamine and of a saturated or unsaturated, linear or branched C₈-C₃₀ fatty acid, the alkanolamine and/or the fatty acid being optionally oxyalkylenated and more particularly oxyethylenated with 1 to 50 mol of ethylene oxide.

Preferably, the fatty acid amides are chosen from amides of a C₂-C₁₀ alkanolamine and of a C₁₄-C₃₀ fatty acid, and even more preferentially from amides of a C₂-C₁₀ alkanolamine and of a C₁₄-C₂₂ fatty acid.

Advantageously, the fatty acid amide is chosen from:
coconut acid monoisopropanolamide, such as the amide sold under the trade name Empilan CLS by the company Huntsman,
oleic acid diethanolamide, such as the amide sold under the trade name Mexanyl® GT by the company Chimex,
myristic acid monoethanolamide, such as the amide sold under the trade name Comperlan® MM by the company Cognis,
soybean fatty acid diethanolamide, such as the amide sold under the trade name Comperlan® VOD by the company Cognis,
stearic acid ethanolamide, such as the amide sold under the trade name Monamid® S by the company Uniqema,
oleic acid monoisopropanolamide, such as the amide sold under the trade name Witcamide® 61 by the company Witco,
linoleic acid diethanolamide, such as the amide sold under the trade name Purton® SFD by the company Zschimmer Schwarz,
stearic acid monoethanolamide, such as the amide sold under the trade name Monamid® 972 by the company ICI/Uniqema,
behenic acid monoethanolamide, such as the amide sold under the trade name Incromide® BEM from Croda,
isostearic acid monoisopropanolamide, such as the amide sold under the trade name Witcamide® SPA by the company Witco,
erucic acid diethanolamide, such as the amide sold under the trade name erucic acid diethanolamide by the company Stéarineries Dubois,
ricinoleic acid monoethanolamide, such as the amide sold under the trade name ricinoleic monoethanolamide by the company Stéarineries Dubois,
rapeseed fatty acid amide containing 4 mol of ethylene oxide, such as the product sold under the name Amidet N by the company Kao.

Oxyalkylenated Fatty Acid Esters

The nonionic thickener(s) may be chosen from oxyalkylenated derivatives of fatty acid esters or of fatty alcohol ethers.

Oxyalkylenated derivatives of fatty acid esters that may especially be mentioned include ethoxylated alkyl or acyl derivatives of polyols, which may in particular be oxyethylenated derivatives of C₆-C₃₀ fatty acid esters or of C₆-C₃₀ fatty alcohol ethers, and of polyols such as glycerol, sorbitol, glucose, pentaerythritol or polyethylene glycol, more particularly polyethylene glycol, these oxyethylenated derivatives generally comprising from 50 to 500 oxyethylene groups and preferably from 100 to 300 oxyethylene groups.

Examples of compounds of this type that may be mentioned include ethylene glycol stearate, polyethylene glycol distearate comprising 150 oxyethylene groups (150 OE), oxyethylenated (200 OE) glyceryl stearate, such as the product sold under the name Simulsol 220 TM® by the company SEPPIC, oxyethylenated (150 OE) pentaerythrityl tetrastearate, such as the product sold under the name Crothix® by the company Croda, oxyethylenated (120 OE) methylglucose dioleate, such as the product sold under the name Glucamate DOE-120 Vegetal® by the company Amerchol, oxyethylenated (160 OE) sorbitan triisostearate such as the product sold under the name Rheodol TW IS399C by the company Kao Chemicals, and oxyethylenated (55 ethylene oxide) propylene glycol oleate, such as the product sold under the reference Antil 141 Liquid by the company Evonik Goldschmidt, and mixtures thereof.

In one variant of the invention, the oxyethylenated fatty acid esters are of formula (A) below:

R₁—CO(X)ₙ—(OCH₂CH₂)ₘ—O—(CO)ₚ—R₂ (A)

X denoting a linear or branched C₁-C₄ alkylene radical, and preferably the radical having the following formula:

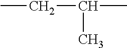

n denoting 0 or 1,

p denoting 0 or 1,

m ranging from 50 to 200, and

R₁ denoting a linear or branched C₉-C₂₉ alkyl or alkenyl radical and R₂ denoting a hydrogen atom or a linear or branched C₉-C₂₉ alkyl or alkenyl radical.

More particularly, among these esters, polyethylene glycol distearate comprising 150 oxyethylene groups (150 OE) is preferred.

Thickening Polymers

The thickening polymers of the invention are different from the amides and esters already described and also from products resulting merely from the condensation of an alkylene oxide with an alcohol, an ester or an amide.

In particular, the nonionic thickening polymers are non-associative.

According to the invention, the term “non-associative thickening polymer” means a thickening polymer not simultaneously comprising at least one C₈-C₃₀ fatty chain and at least one hydrophilic unit.

Preferably the thickening polymers of the invention are non ionic.

The nonionic non-associative thickening polymers according to the invention may be of natural or synthetic origin. They are chosen especially from:

(i) nonionic homopolymers and copolymers containing ethylenically unsaturated monomers of ester and/or amide type,

(ii) vinylpyrrolidone homopolymers or copolymers,

(iii) polysaccharides.

Among the nonionic homopolymers or copolymers containing ethylenically unsaturated monomers of ester and/or amide type that may be mentioned are polyamides, especially the products sold under the names: Cyanamer P250 by the company Cytec (polyacrylamide); methyl methacrylate/ethylene glycol dimethacrylate copolymers (PMMA MBX-8C by the company US Cosmetics); butyl methacrylate/methyl methacrylate copolymers (Acryloid B66 by the company Röhm & Haas); polymethyl methacrylate (BPA 500 by the company Kobo).

The vinylpyrrolidone homopolymers or copolymers are chosen especially from crosslinked vinylpyrrolidone homopolymers such as the Polymer ACP-10 sold by ISP.

The thickening polysaccharides are especially chosen from glucans, modified or unmodified starches (such as those derived, for example, from cereals, for instance wheat, corn or rice, from vegetables, for instance yellow pea, and tubers, for instance potato or cassava), amylose, amylopectin, glycogen, dextrans, celluloses and derivatives thereof (methylcelluloses, hydroxyalkylcelluloses, ethylhydroxyethylcelluloses), mannans, xylans, lignins, arabans, galactans, galacturonans, chitin, chitosans, glucuronoxylans, arabinoxylans, xyloglucans, glucomannans, pectic acids and pectins, arabinogalactans, carrageenans, agars, gum arabics, gum tragacanths, ghatti gums, karaya gums, carob gums, galactomannans such as guar gums and nonionic derivatives thereof (hydroxypropyl guar), and mixtures thereof.

In general, the compounds of this type that may be used in the present invention are chosen from those described especially in Kirk-Othmer's Encyclopedia of Chemical Technology, Third Edition, 1982, volume 3, pp. 896-900, and volume 15, pp. 439-458, in Polymers in Nature by E. A. MacGregor and C. T. Greenwood, published by John Wiley & Sons, Chapter 6, pp. 240-328, 1980, and in Industrial Gums—Polysaccharides and their Derivatives, edited by Roy L. Whistler, Second Edition, published by Academic Press Inc., the content of these three publications being entirely included in the present patent application by way of reference.

Starches, guar gums and celluloses and derivatives thereof will preferably be used.

The polysaccharides as mentioned before can be modified or unmodified.

The unmodified guar gums are, for example, the products sold under the name Vidogum GH 175 by the company Unipectine and under the names Meypro-Guar 50 and Jaguar C by the company Rhodia Chimie.

The modified nonionic guar gums are especially modified with C1-C6 hydroxyalkyl groups.

Among the hydroxyalkyl groups that may be mentioned, for example, are hydroxymethyl, hydroxyethyl, hydroxypropyl and hydroxybutyl groups.

These guar gums are well known in the prior art and can be prepared, for example, by reacting the corresponding alkene oxides such as, for example, propylene oxides, with the guar gum so as to obtain a guar gum modified with hydroxypropyl groups.

The degree of hydroxyalkylation, which corresponds to the number of alkylene oxide molecules consumed by the number of free hydroxyl functions present on the guar gum, preferably ranges from 0.4 to 1.2.

Such nonionic guar gums optionally modified with hydroxyalkyl groups are sold, for example, under the trade names Jaguar HP8, Jaguar HP60 and Jaguar HP120, Jaguar DC 293 and Jaguar HP 105 by the company Rhodia Chimie or under the name Galactasol 4H4FD2 by the company Aqualon.

Among the celluloses that are especially used are hydroxyethylcelluloses and hydroxypropylcelluloses. Mention may be made of the products sold under the names Klucel EF, Klucel H, Klucel LHF, Klucel MF and Klucel G by the company Aqualon, and Cellosize Polymer PCG-10 by the company Amerchol.

In a preferred embodiment, the thickening agents used in the composition of the present invention are nonionic.

In a preferred embodiment, the thickening agents used in the composition are non ionic agents chosen from fatty alcohols, non ionic non-associative polymers and mixture thereof.

Particularly, the thickening agents are chosen from fatty alcohols and polysaccharides and more particularly from fatty alcohols and non ionic polysaccharides.

The thickening agents used in the present invention may be present in said composition in an amount of from 0.1 wt % to 15 by wt %, more preferably from 0.5 to 10 by wt %, preferably from 1 by wt % to 8 by wt %, relative to the total weight of the composition.

The compositions of the invention preferably comprise water or a mixture of water and one or more organic solvents chosen from C₁-C₄ lower alcohols, such as ethanol, isopropanol, tert-butanol or n-butanol; polyols such as glycerol, propylene glycol and polyethylene glycols; and mixtures thereof.

The composition according to the invention comprises an amount of water which is generally greater than or equal to 5% by weight, relative to the total weight of the composition, preferably an amount of water greater than or equal to 20% by weight relative to the total weight of the composition.

Preferably, the amount of water in the composition according to the invention is less than or equal to 95% and preferentially less than or equal to 90% by weight relative to the total weight of the composition. The organic solvents may be present in a concentration ranging from 0.1% to 40% and better still from 1% to 20% by weight relative to the total weight of the composition.

The pH of the compositions according to the invention generally ranges from 3 to 9, most preferably from 4 to 7.

The composition according to the invention may also comprise one or more standard additives that are well known in the art, chosen from surfactants other than cationic surfactants previously described, particularly non ionic surfactants, moisturizers; emollients, plasticizers, permanent or temporary dyes, fragrances, peptizers, preserving agents, active agents, ceramides or pseudoceramides; oils and waxes, vitamins or provitamins; pH stabilizers, preserving agents; proteins, sequestrants; solubilizers; reducing agents or antioxidants; oxidizing agents; basifying agents, acidifying agents, anticorrosion agents and non-thickening cationic polymers, and mixtures thereof.

A person skilled in the art will take care to select the optional standard additives and the amount thereof such that they do not harm the properties of the compositions of the present invention.

These standard additives are generally present in the composition according to the invention in an amount ranging from 0 to 20% by weight relative to the total weight of the composition.

The composition according to the invention may be in the form of an emulsion. More specifically, it may be either in the form of an oil-in-water emulsion, with the continuous phase being the aqueous phase, or in the form of a water-in-oil emulsion, with the continuous phase being the fatty phase.

The compositions in accordance with the invention may be used for conditioning keratin fibres, in particular the hair, for example as hair conditioners.

Preferably, the compositions of the invention are rinse-off or leave-in hair conditioners.

According to one embodiment of the present invention, the composition comprises:

(i) one or more amino silicones,

(ii) one or more cationic surfactants different from (i),

(iii) one or more associative polymers chosen from the cationic amphiphilic polymers,

(iv) one or more thickening agents different from the associative polymers (iii) chosen from the non ionic thickening agents.

According to this embodiment, the cationic surfactants preferably are chosen from quaternary ammonium salts and more preferably chosen from quaternary ammonium salts of formula (III) and quaternary ammonium salts comprising at least one ester function, especially those selected from those corresponding to the formula (VI).

According to this embodiment, the amino silicones are preferably chosen from multiblock polyalkylenated amino silicones.

According to one other embodiment of the present invention, the composition comprises:

(i) one or more amino silicones,

(ii) one or more cationic surfactants different from (i),

(iii) one or more associative polymers chosen from the cationic amphiphilic polymers chosen from quaternized cellulose, particularly hydroxyethylcellulose modified with groups comprising at least one fatty chain having at least 8 carbon atoms,

(iv) one or more nonionic thickening agents different from the associative polymers (iii) chosen from fatty alcohols and non-associative polymers.

Preferably, the composition comprises:

(I) one or more amino silicones chosen from multiblock polyalkylenated amino silicones,

(ii) one or more cationic surfactants chosen from quaternary ammonium salts and more preferably chosen from quaternary ammonium salts of formula (III),

(iii) one or more associative polymers chosen from the cationic amphiphilic polymers chosen quaternized cellulose, particularly hydroxyethylcellulose modified with groups comprising at least one fatty chain having at least 8 carbon atoms,

(iv) one or more nonionic thickening agents different from the associative polymers (iii) chosen from fatty alcohols, non-associative polymers and mixtures thereof, especially fatty alcohols and non ionic polysaccharides.

Another subject of the present invention is a process for treating keratin fibres, such as the hair, which consists in applying a composition as described above on the said fibres.

The compositions described above may be used on any type of hair: light or dark hair, natural hair or hair that has undergone a cosmetic treatment such as permanent waving, dyeing, bleaching or relaxing.

In a preferred embodiment, the composition of the present invention is applied on sensitized hair.

The application to the hair of the composition according to the invention may be performed, for example, using a comb, a fine brush, a coarse brush or with the fingers.

According to one particular embodiment of the invention, the application of the composition is followed by drying at room temperature or at a temperature above 40° C.

The drying may be performed immediately after the application or after a leave-on time that may range from 1 minute to 30 minutes.

Preferably, the hair is dried, in addition to using a supply of heat, with a flow of air. This flow of air during drying makes it possible to improve the individualization of the coating.

During drying, a mechanical action may be exerted on the locks, such as combing, brushing or by running the fingers through the hair.

The drying step of the process of the invention may be performed with a hood, a hair dryer, a straightening iron, etc.

When the drying step is performed with a hood or a hair dryer, the drying temperature is between 40 and 110° C. and preferably between 50 and 90° C.

When the drying step is performed with a straightening iron, the drying temperature is between 110 and 230° C. and preferably between 130 and 200° C.

The invention also relates to the use of the composition according to the invention as a hair conditioner, preferably used after the application of a shampoo or shampoo and other conditioner.

The examples below are given as illustrations of the present invention.

### EXAMPLE

Concentrations are given as active raw material.

| | Concentration |
|---|---|
| Nom INCl US | (active raw material) |
| POLYQUATERNIUM-67 | 0.237 |
| HYDROXYETHYLCELLULOSE | 0.5 |
| CHLORHEXIDINE DIGLUCONATE | 0.04 |
| CETEARYL ALCOHOL | 3.0 |
| CITRIC ACID | 0.08 |
| BEHENTRIMONIUM CHLORIDE | 3.16 |
| PEG-40/PPG-8 | 1.2 |
| METHYLAMINOPROPYL/ | |
| HYDROXYPROPYL | |
| DIMETHICONE COPOLYMER | |
| FRAGRANCE | 0.4 |
| WATER | Qsp 100 |

The composition is applied on hair washed by a shampoo. After final rinsing, hair is softer, smoother and suppler and the number of splits ends is also reduced.

1.-25. (canceled)

26. A composition comprising:
(i) at least one amino silicone,
(ii) at least one cationic surfactant different from (i),
(iii) at least one associative polymer, and
(iv) at least one thickening agent different from the associative polymer (iii).

27. The composition according to claim 26, wherein the amino silicone is chosen from:
(a) compounds corresponding to formula (I) below:
(R¹)ₐ(T)₃₋ₐ-Si[OSi(T)₂]ₙ-[OSi(T)_{b}(R¹)_{2-b}]ₘ—OSi(T)₃₋ₐ-(R¹)ₐ (I)
wherein:
T is a hydrogen atom or a phenyl, hydroxyl (—OH), C₁-C₈ alkyl radical, methyl, C₁-C₈ alkoxy, or methoxy,
a denotes the number 0 or an integer from 1 to 3,
b denotes 0 or 1,
m and n are numbers such that the sum (n+m) can range from 1 to 2000, wherein n denotes a number from 0 to 1999 and m denotes a number from 1 to 2000;
R¹ is a monovalent radical of formula —C_{q}H_{2q}L wherein q is a number from 2 to 8 and L is an optionally quaternized amino group chosen from the following groups:
—N(R²)—CH₂—CH₂—N(R²)₂;
—N(R²)₂; —N⁺(R²)₃ Q⁻;
—N⁺(R²) (H)₂ Q⁻;
—N⁺(R²)₂HQ⁻; or
—N(R²)—CH₂—CH₂—N⁺(R²)(H)₂ Q⁻,
wherein R² denotes a hydrogen atom, a phenyl, a benzyl or a saturated monovalent hydrocarbon-based radical, and Q⁻ represents a halide ion, fluoride, chloride, bromide or iodide,
(b) the compounds corresponding to formula (II) below:
wherein: 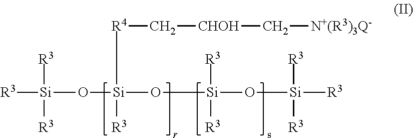
R³ represents a C₁-C₁₈ monovalent hydrocarbon-based radical, a C₁-C₁₈ alkyl or C₂-C₁₈ alkenyl radical;
R⁴ represents a divalent hydrocarbon-based radical, a C₁-C₁₈ alkylene radical or a divalent C₁-C₁₈;
Q⁻ is a halide ion;
r represents a mean statistical value from 2 to 20;
s represents a mean statistical value from 20 to 200,
(c) the quaternary ammonium silicones of formula (III): 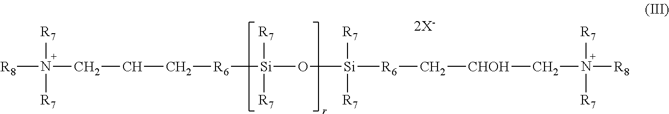
wherein:
R₇, which may be identical or different, represent a monovalent hydrocarbon-based radical containing from 1 to 18 carbon atoms, a C₁-C₁₈ alkyl radical, a C₂-C₁₈ alkenyl radical or a ring comprising 5 or 6 carbon atoms;
R₆ represents a divalent hydrocarbon-based radical, a C₁-C₁₈ alkylene radical or a divalent C₁-C₁₈;
R₈, which may be identical or different, represent a hydrogen atom, a monovalent hydrocarbon-based radical containing from 1 to 18 carbon atoms, a C₂-C₁₈ alkenyl radical or a radical —R₆—NHCOR₇;
X⁻ is an anion or an organic acid salt;
r represents a mean statistical value from 2 to 200;
(d) the amino silicones of formula (IV) below: 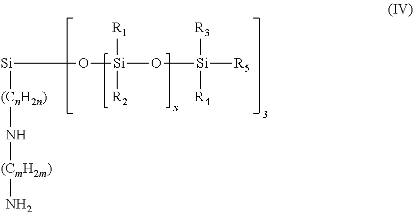
wherein:
R₁, R₂, R₃ and R₄, which may be identical or different, denote a C₁-C₄ alkyl radical or a phenyl group,
R₅ denotes a C₁-C₄ alkyl radical or a hydroxyl group,
n is an integer ranging from 1 to 5,
m is an integer ranging from 1 to 5, and
wherein x is chosen such that the amine number is between 0.01 and 1 meq/g;
(e) multiblock polyoxyalkylenated amino silicones, of the type (AB)n, A being a polysiloxane block and B being a polyoxyalkylene block comprising at least one amine group; or
(f) multiblock polyoxyalkylenated amino silicones, of the type (AB)n, A being a polysiloxane block and B being a polyoxyalkylene block comprising at least one amine group, silicones optionally consisting of repeating units having the following general formulae:
[—(SiMe₂O)ₓSiMe₂-R—N(R″)—R′—O(C₂H₄O)ₐ(C₃H₆O)_{b}—R—N(H)—R—]
or alternatively
[—(SiMe₂O)ₓSiMe₂-R—N(R″)—R′—O(C₂H₄O)ₐ(C₃H₆O)_{b}—]
wherein:
a is an integer greater than or equal to 1;
b is an integer between 0 and 200;
x is an integer ranging from 1 to 10 000;
R″ is a hydrogen atom or a methyl;
R, which may be identical or different, represent a linear or branched C₂-C₁₂ divalent hydrocarbon-based radical, optionally comprising one or more heteroatoms, an ethylene radical, a linear or branched propylene radical, a linear or branched butylene radical, or a —CH₂CH₂CH₂OCH(OH)CH₂— radical;
R′, which may be identical or different, represent a linear or branched C₂-C₁₂ divalent hydrocarbon-based radical, optionally comprising one or more heteroatoms, an ethylene radical, a linear or branched propylene radical, a linear or branched butylene radical, or a —CH₂CH₂CH₂OCH(OH)CH₂— radical.

28. The composition according to claim 26, wherein the amino silicone is present in a content ranging from 0.1% to 15% by weight, relative to the total weight of the composition.

29. The composition according to claim 26, wherein the cationic surfactant is chosen from:
salts of optionally polyoxyalkylenated primary, secondary or tertiary fatty amines;
quaternary ammonium salts;
behenyltrimethylammonium, distearyldimethylammonium, cetyltrimethylammonium, benzyldimethylstearylammonium chlorides;
palmitylamidopropyltrimethylammonium or stearamidopropyldimethyl(myristyl acetate)-ammonium chlorides;
quaternary ammonium salts chosen from:
those that have the general formula (VI) below: 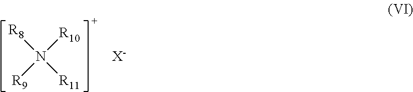
wherein the radicals R₈ to R₁₁, which may be identical or different, represent a linear or branched aliphatic radical comprising from 1 to 30 carbon atoms, or an aromatic radical; X is an anion chosen from halides, phosphates, acetates, lactates, (C₂-C₆)alkyl sulfates, or alkyl or alkylaryl sulfonates;
quaternary ammonium salts of imidazoline;
di- or triquaternary ammonium salts, in particular of formula (VIII): 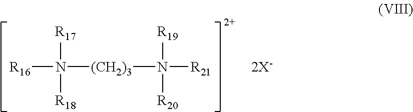
wherein R₁₆ denotes an alkyl radical containing approximately from 16 to 30 carbon atoms, which is optionally hydroxylated and/or interrupted with one or more oxygen atoms and optionally branched, R₁₇ is selected from an alkyl radical containing from 1 to 4 carbon atoms or a group (R₁₆ₐ)(R₁₇ₐ)(R₁₈ₐ)N₊—(CH₂)₃—, R₁₆ₐ, R₁₇ₐ, R₁₈ₐ, R₁₈, R₁₉, R₂₀ and R₂₁, which may be identical or different, are selected from hydrogen and an alkyl radical containing from 1 to 4 carbon atoms, and X⁻ is an anion selected from the group of halides, acetates, phosphates, nitrates and methyl sulfates;
quaternary ammonium salts containing at least one ester function of formula (IX) below: 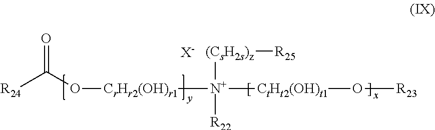
wherein:
R₂₂ is chosen from linear or branched C₁-C₆ alkyl radicals and C₁-C₆ hydroxyalkyl or dihydroxyalkyl radicals;
R₂₃ is chosen from:
the group 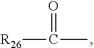
radicals R₂₇, which are linear or branched, saturated or unsaturated C₁-C₂₂ hydrocarbon-based radicals,
a hydrogen atom,
R₂₅ is chosen from:
the group 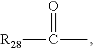
radicals R₂₉, which are linear or branched, saturated or unsaturated C₁-C₆ hydrocarbon-based radicals,
a hydrogen atom,
R₂₄, R₂₆ and R₂₈, which may be identical or different, are chosen from linear or branched, saturated or unsaturated C₇-C₂₁ hydrocarbon-based radicals;
r, s and t, which may be identical or different, are integers ranging from 2 to 6;
y is an integer ranging from 1 to 10;
x and z, which may be identical or different, are integers ranging from 0 to 10;
r₁ or t₁=0 or 1, r₂+r₁=2r and t₂+t₁=2t,
X⁻ is a simple or complex, organic or mineral anion;
wherein the sum x+y+z is from 1 to 15, that when x is 0, then R₂₃ denotes R₂₇ and that when z is 0, then R₂₅ denotes R₂₉; or
combinations thereof.

30. The composition according to claim 26, wherein the cationic surfactant is present in an amount ranging from 0.1% to 20%, relative to the total weight of the composition.

31. The composition according to claim 26, wherein the associative polymer is chosen from amphiphilic polymers.

32. The composition according to claim 26, wherein the associative polymer is chosen from anionic, non ionic, cationic or amphoteric associative polymers.

33. The composition according to claim 26, wherein the associative polymer is an anionic amphiphilic associative polymer chosen from:
(I) anionic associative polymers comprising at least one hydrophilic unit and at least one fatty-chain allyl ether unit, including a hydrophilic unit comprising an ethylenic unsaturated anionic monomer, vinylcarboxylic acid, acrylic acid, methacrylic acid, or mixtures thereof, wherein the fatty-chain allyl ether unit of which corresponds to the monomer of formula (X) below:
CH₂—CR′CH₂OBₙR (X)
wherein:
R′ is chosen from a hydrogen atom and CH₃;
B is an ethyleneoxy radical;
n is an integer ranging from 0 to 100;
R is a hydrocarbon-based radical chosen from alkyl, arylalkyl, aryl, alkylaryl, or cycloalkyl radicals, having from 8 to 30 carbon atoms,
(II) anionic associative polymers comprising at least one unsaturated olefinic carboxylic acid hydrophilic unit, and at least one unsaturated carboxylic acid (C₁₀-C₃₀)alkyl ester hydrophobic unit,
(III) maleic anhydride/C₃₀-C₃₈ α-olefin/alkyl maleate terpolymers,
(IV) acrylic terpolymers comprising:
(a) 20% to 70% by weight of a carboxylic acid containing α, β-monoethylenic unsaturation,
(b) 20% to 80% by weight of a non-surfactant monomer containing α, β-monoethylenic unsaturation and being other than (a),
(c) 0.5% to 60% by weight of a non-ionic monourethane which is the product of reaction of a monohydric surfactant with a monoisocyanate containing monoethylenic unsaturation,
(V) copolymers comprising among their monomers a carboxylic acid containing α, β-monoethylenic unsaturation and an ester of a carboxylic acid containing α, β-monoethylenic unsaturation and of an oxyalkylenated fatty alcohol.

34. The composition according to claim 26, wherein the associative polymer is a non ionic amphiphilic associative polymer chosen from:
(1) celluloses modified with groups comprising at least one fatty chain;
(2) hydroxypropyl guars modified with groups comprising at least one fatty chain,
(3) copolymers of C₁-C₆ alkyl methacrylates, acrylates, or amphiphilic monomers comprising at least one fatty chain,
(4) copolymers of hydrophilic methacrylates, acrylates, or hydrophobic monomers comprising at least one fatty chain,
(5) polyurethane polyethers comprising in their chain both hydrophilic blocks of polyoxyethylenated nature and hydrophobic blocks, which may be aliphatic sequences alone and/or cycloaliphatic and/or aromatic sequences, or
(6) polymers with an aminoplast ether backbone containing at least one fatty chain.

35. The composition according to claim 26, wherein the associative polymer is a cationic amphiphilic associative polymer chosen from:
(A) cationic associative amphiphilic polyurethanes,
(B) quaternized cellulose modified with groups comprising at least one fatty chain, including alkyl, arylalkyl or alkylaryl groups comprising at least 8 carbon atoms,
(C) cationic associative poly(vinyllactam) polymer, or
(D) the cationic polymer(s) obtained by polymerization of a monomer mixture comprising one or more vinyl monomers substituted with one or more amino groups, one or more hydrophobic nonionic vinyl monomers, or one or more associative vinyl monomers.

36. The composition according to claim 26, wherein the associative polymer is an amphoteric amphiphilic associative polymer comprising:
1) at least one monomer of formula (XVIa) or (XVIb): 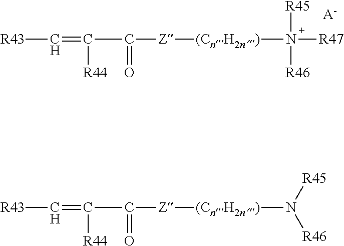
wherein R₄₃ and R₄₄, which may be identical or different, are chosen from a hydrogen atom and methyl; R₄₅, R₄₆, and R₄₇, which may be identical or different, are chosen from linear and branched alkyl radicals having from 1 to 30 carbon atoms;
Z″ is chosen from an NH group or an oxygen atom;
n′″ is an integer from 2 to 5; and
A⁻ is an anion derived from an acid chosen from organic and mineral acids, methosulphate anions or halides;
2) at least one monomer of formula (XVII): 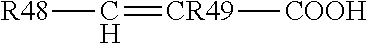
wherein R₄₈ and R₄₉, which may be identical or different, are chosen from a hydrogen atom and methyl; and
3) at least one monomer of formula (XVIII): 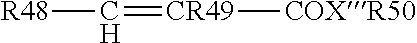
wherein R₄₈ and R₄₉, which may be identical or different, are chosen from a hydrogen atom and methyl, X′″ is chosen from oxygen and nitrogen, and R₅₀ is chosen from linear and branched alkyl radicals having from 1 to 30 carbon atoms;
wherein at least one of the monomers of formula (XVIa), (XVVb) and (XVII) comprises at least one fatty chain.

37. The composition according to claim 26, wherein the associative polymer is a cationic amphiphilic associative polymer chosen from quaternized hydroxyethylcelluloses modified with at least one alkyl group having 10 to 22 carbon atoms.

38. The composition according to claim 26, wherein the associative polymer is present in an amount ranging from 0.01% to 5%, relative to the total weight of the composition.

39. The composition according to claim 26, wherein the thickening agent different from (iii) is chosen from non ionic thickening agents, fatty alcohols, fatty acid amides, oxyalkylenated fatty acid esters, nonionic thickening polymers, polysaccharides, or mixtures thereof.

40. The composition according to claim 26, wherein the thickening agent is chosen from:
fatty alcohols chosen from lauryl alcohol, cetyl alcohol, stearyl alcohol, oleyl alcohol, behenyl alcohol, linoleyl alcohol, undecylenyl alcohol, palmitoleyl alcohol, arachidonyl alcohol, erucyl alcohol, or mixtures thereof,
fatty acid amides chosen from amides of a C₂-C₁₀ alkanolamine and of a C₁₄-C₃₀ fatty acid,
oxyalkylenated derivatives of fatty acid esters chosen from the compounds of formula (A) below:
R₁—CO(X)ₙ—(OCH₂CH₂)ₘ—O—(CO)ₚ—R₂ (A)
X denoting a linear or branched C₁-C₄ alkylene radical, and the radical having the following formula: 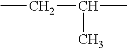
n denoting 0 or 1,
p denoting 0 or 1,
m ranging from 50 to 200, and
R₁ denoting a linear or branched C₉-C₂₉ alkyl or alkenyl radical and R₂ denoting a hydrogen atom or a linear or branched C₉-C₂₉ alkyl or alkenyl radical; and
non ionic non associative polymers chosen from:
(i) nonionic homopolymers and copolymers containing ethylenically unsaturated monomers of ester and/or amide type,
(ii) vinylpyrrolidone homopolymers or copolymers, or
(iii) polysaccharides.

41. The composition according to claim 26, wherein the thickening agent is present in an amount ranging from 0.1% to 15%, relative to the total weight of the composition.

42. A process for treating the hair, comprising applying to the hair a composition comprising:
(i) at least one amino silicone,
(ii) at least one cationic surfactant different from (i),
(iii) at least one associative polymer, and
(iv) at least one thickening agent different from the associative polymer (iii).

43. The process according to claim 42, further comprising, after applying to the hair the composition, drying the hair at room temperature or at a temperature above 40° C.

44. The process according claim 43, wherein the drying step is performed with a hood, a hair dryer or a straightening iron.

45. The process according to claim 42, wherein the composition is applied to the hair after application of a shampoo.
